(19) United States
(12) Patent Application Publication
(10) Pub. No.: US 2025/0174333 A1
(43) Pub. Date: May 29, 2025

(54) PROVIDING DISORDER TREATMENT ESTIMATES BASED ON DISORDER DATA

(71) Applicant: Align Technology, Inc., San Jose, CA (US)

(72) Inventors: Svetozar Grigorov Hubenov, Luzern (CH); Leela Mohan Parvathaneni, San Jose, CA (US); Sravani Gurijala, Apex, NC (US); Lakshmi Amirneni, Apex, NC (US); Bandhavi Nalajala, Durham, NC (US); Shalini Akur, Sam Ramon, CA (US); Teja Atluri, San Francisco, CA (US); Mario Lanzas, Union City, CA (US); Julijana Taskovic, Phoenix, AZ (US); Ritvik Bansal, San Francisco, CA (US); Marina Sharm, San Jose, CA (US); Lavanya Reddy Reddy, Pleasanton, CA (US)

(21) Appl. No.: 18/959,402

(22) Filed: Nov. 25, 2024

Related U.S. Application Data
(60) Provisional application No. 63/603,052, filed on Nov. 27, 2023.
(60) Provisional application No. 63/666,354, filed on Jul. 1, 2024.

(51) Int. Cl. G16H 20/40; G16H 10/60; G16H 30/40; G16H 50/20

(52) U.S. Cl. CPC G16H 20/40; G16H 10/60; G16H 30/40; G16H 50/20

(57) ABSTRACT

A method includes determining, by a processing device, a plurality of treatment providers for providing a dental treatment to a user and providing identification of the plurality to a first user device. The method further includes receiving image data of dentition of the user from the first user device. The method further includes receiving a user selection of a first treatment provider and a second treatment provider. The method further includes providing the image data to a second and third device associated with the first treatment provider and second treatment provider. The method further includes obtaining first information of a first recommended treatment for the dentition and second information of a second recommended treatment for the dentition from the first and second treatment providers. The method further includes providing the first information and the second information of the second recommended treatment to the first user device.

### RELATED APPLICATIONS

This patent application claims the benefit under 35 U.S.C. § 119(e) of U.S. Provisional Application No. 63/603,052, filed Nov. 27, 2023, and of U.S. Provisional Application No. 63/666,354, filed Jul. 1, 2024, both of which are incorporated by reference herein.

### TECHNICAL FIELD

Embodiments of the present invention relate to the field of dentistry, and in particular to methods of providing disorder treatment estimates based on disorder data.

### BACKGROUND

When a dentist or orthodontist is engaging with current and/or potential patients, it is often helpful to generate data indicative of dental arches of the patients. For example, it may be helpful to view data of dentition in order to generate estimates of recommended treatment. However, often systems for sending and receiving disorder data may be complex or may not have been implemented. Determining estimates of treatment may include examination of a patient or patient data by a treatment provider or practitioner. Determining estimates of treatment may include examination of private patient data by a treatment provider.

### SUMMARY

The following is a simplified summary of the disclosure in order to provide a basic understanding of some aspects of the disclosure. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In one aspect of the present disclosure, a method includes determining, by a processing device, a plurality of treatment providers for providing a dental treatment to a user. The method further includes providing to a first user device identification of the plurality of treatment providers. The method further includes receiving image data of dentition of the user from the first user device. The method further includes receiving a first user selection of a first treatment provider of the plurality of treatment providers. The method further includes receiving a second user selection of a second treatment provider of the plurality of treatment providers from the first user device. The method further includes providing the image data to a second device associated with the first treatment provider and a third device associated with the second treatment provider. The method further includes obtaining first information of a first recommended treatment for the dentition from the second device of the first treatment provider. The method further includes obtaining second information of a second recommended treatment for the dentition from the third device of the second treatment provider. The method further includes providing the first information of the first recommended treatment and the second information of the second recommended treatment to the first user device.

In another aspect of the present disclosure, a method includes obtaining, by a processing device, identification of a plurality of treatment providers. The method further includes obtaining user input. The user input is indicative of a first disorder, a first selection of a first treatment provider of the plurality of treatment providers, and a second selection of a second treatment provider of the plurality of treatment providers. The method further includes providing the user input to a server device. The method further includes receiving, from the server device, first information of a first recommended treatment in association with the first disorder and the first treatment provider. The method further includes receiving second information of a second recommended treatment in association with the first disorder and the second treatment provider. The method further includes obtaining a first selection of the first recommended treatment. The method further includes providing an indication of the first recommended treatment based on the third selection of the first recommended treatment to the server device.

In another aspect of the present disclosure, a method includes obtaining, by a processing device, from a first user device, a first user request for identification of a first treatment provider. The method further includes determining a treatment provider. The method further includes providing identification of the treatment provider to the first user device. The method further includes receiving from the first user device first user data. The first user data includes data indicative of dentition of the user. The first data includes a second user request for a first treatment plan associated with the dentition and the treatment provider. The method further includes providing the first user data to a second device associated with the treatment provider. The method further includes obtaining, from the second device, information of a first recommended treatment in association with the first user data and the treatment provider. The method further includes providing to the first user device the information of the first recommended treatment.

In another aspect of the present disclosure, a method includes determining a first set of payment sources for a dental treatment. The method further includes determining one or more treatment providers for providing the dental treatment to a first user. The method further includes providing to a first user device identification of the one or more treatment providers. The method further includes obtaining information of a first recommended treatment for dentition of the first user from the first treatment provider. The information includes estimated cost of treatment incorporating the first set of payment sources. The method further includes providing the first information of the first recommended treatment to the first user device.

In another aspect of the present disclosure, a method includes determining a first set of payment sources in association with a first healthcare service. The method further includes determining one or more treatment provider for providing the healthcare service to a first user. The method further includes providing to a first user device identification of the one or more treatment providers. The method further includes obtaining first information of a first recommended treatment plan in association with the first healthcare service. The first information includes an estimated cost of treatment in accordance with the first recommended treatment plan incorporating the first set of payment sources. The method further includes providing the first information to the first user device.

### BRIEF DESCRIPTION OF THE DRAWINGS

The present disclosure is illustrated by way of example, and not by way of limitation in the figures of the accompanying drawings.

FIG. 1 is a block diagram illustrating an exemplary system architecture for providing tools for communication and management of aspects of treatment, according to some embodiments.

FIG. 2 is a diagram of a flow for providing treatment estimates to a user, according to some embodiments.

FIG. 3A depicts an example graphical user interface (GUI) of a user device for providing disorder data, according to some embodiments.

FIG. 3B depicts a GUI for enabling a treatment provider to provide a treatment estimate, according to some embodiments.

FIG. 3C illustrates workflows for training and implementing one or more machine learning models for performing operations associated with treatment estimates, according to some embodiments.

FIG. 3D is a block diagram of a flow for obtaining a treatment estimate, including multiple payment sources, according to some embodiments.

FIG. 4A is a flow diagram of a method providing an estimate for treatment to a user, according to some embodiments.

FIG. 4B is a flow diagram of a method for providing disorder data and receiving treatment estimates based on the disorder data, according to some embodiments.

FIG. 4C is a flow diagram of a method for providing recommended treatment information to a user, according to some embodiments.

FIG. 4D is a flow diagram of a method for generating treatment estimates, according to some embodiments.

FIG. 4E is a flow diagram of a method for applying payment sources to a dental treatment, according to some embodiments.

FIG. 4F is a flow diagram of a method for applying payment sources to healthcare services, according to some embodiments.

FIG. 5A illustrates a tooth repositioning system including a plurality of appliances, in accordance with some embodiments.

FIG. 5B illustrates a method of orthodontic treatment using a plurality of appliances, in accordance with some embodiments.

FIG. 6 illustrates a method for designing an orthodontic appliance to be produced by direct fabrication, in accordance with some embodiments.

FIG. 7 illustrates a method for digitally planning an orthodontic treatment and/or design or fabrication of an appliance, in accordance with some embodiments.

FIG. 8 is a block diagram illustrating a computer system, according to some embodiments.

### DETAILED DESCRIPTION

Described herein are technologies related to providing provisional treatment estimates for one or more disorders to a user. Recommended or provisional treatment estimates may include evaluation of disorders by a treatment provider. Providing provisional treatment estimates may include evaluation of patient or potential patient data by a treatment provider. Providing provisional treatment estimates may include evaluation of multiple disorders. Providing provisional treatment estimates may include evaluation by multiple treatment providers. Providing provisional treatment estimates may include interaction with one or more applications (e.g., of a client device associated with a treatment provider, client device associated with a user, etc.) for exchanging information between a user and a treatment provider.

A user may take steps to receive treatment for one or more disorders, such as prosthodontic or orthodontic treatment for dental arch disorders. Dental arch data may be utilized in treatment of a dental arch. For example, one or more dental malocclusions (e.g., misalignment of teeth) may be treated by an orthodontic treatment plan, which may include collecting and utilizing jaw pair data of a patient. As a further example, generation of a crown or dental implant may be performed based on dental arch data. Dental arch data may include data of one or more teeth (e.g., including size, shape, positioning, orientation, etc.), a group of teeth, an arch, an upper and lower jaw, etc.

Receiving treatment for disorders may include providing information about a patient disorder to a treatment provider and receiving a treatment recommendation from the treatment provider. In some systems, this may include an in-person or virtual appointment between a user and a treatment provider to enable the treatment provider to assess the disorder, generate a proposed treatment plan, etc. In some systems, this may include user engagement with a system associated with the treatment provider, such as a website, application, or other system for providing of disorder data, messaging between the treatment provider and the user, etc.

Enabling user selection of a treatment provider from a set of potential treatment providers may yield additional difficulties in some circumstances. For example, pursuing each potential treatment provider of interest to the user may include separate appointments, engagements with separate communication systems, or the like. In some cases, appointments for initial assessment, generation of estimated treatment plans, or the like, may incur a cost in time and/or money for a user, occupy an appointment slot for a treatment provider, and may not be efficient, convenient, or the like.

Methods and systems of the present disclosure may address one or more of the shortcomings of conventional systems. In some embodiments, one or more communication tools are provided (e.g., via one or more websites, one or more web browsing applications, one or more purpose-built applications, one or more client devices, etc.). The one or more communication tools may include tools available for treatment providers. The one or more communication tools may additionally or alternatively include tools available for other users, e.g., patients or potential patients. The one or more communication tools may enable communication between a user and one or more treatment providers, a treatment provider and one or more users, etc.

In some embodiments, a user tool (e.g., user application) may provide a set of potential treatment providers to a user. The set of potential treatment providers may be provided responsive to a user request. The set of potential treatment providers may be generated based on user selection of one or more disorders for treatment. The set of potential treatment providers may be generated by a server device. The set of potential treatment providers may be generated based on one or more metrics, such as location, frequency, recency, and/or volume of treatments performed by the treatment providers, a type of disorder and/or treatment, user rating, or other metrics.

In some embodiments, the user tool may enable a user to select one or more treatment providers. Selection of one or more treatment providers may be facilitated by allowing a user to filter a set of providers (e.g., filter out providers with less than a target amount of experience with the user's disorder). Selection of one or more treatment providers may be facilitated by allowing a user to reorder a list of providers (e.g., order the list by nearest geographical location to farthest, order the list based on practitioner experience, etc.). Selection of one or more treatment providers may enable a user to choose one or more treatment providers to enable communication with the treatment providers. The user tool may enable the user to provide data indicative of a disorder to the one or more treatment providers. The user tool may assist the user in generating data indicative of the disorder that may be used by the treatment providers in generating a recommended treatment plan, a treatment estimate, or the like. For example, the user tool may guide a user in generating images indicative of a dental arch disorder for examination by the one or more treatment providers. The user tool may enable the user to provide the data indicative of a disorder (e.g., images of a dental arch) to the one or more selected treatment providers.

In some embodiments, a treatment provider tool (e.g., an application, a website, or the like) may be provided. The treatment provider may utilize the tool for communication with one or more users, patients, potential patients, etc. The treatment provider may utilize the tool to receive messages from one or more users, provide messages to one or more users, etc. The treatment provider may utilize the tool to receive data indicative of a user disorder (e.g., images of a dental arch). The treatment provider may utilize the tool to provide treatment plans, treatment estimates (e.g., estimates of treatment cost, treatment duration, or the like), requests for updated or additional disorder information, or the like to one or more users.

In some embodiments, a backend system (e.g., a server system, a treatment communication system, etc.) may manage one-to-one connections, many-to-many connections and/or one-to-many connections between treatment providers, users (e.g., recipients and potential recipients of treatment), or the like. For example, a first user may be in communication with a number of treatment providers. The first user may be in communication with multiple sets of treatment providers, e.g., treatment providers associated with more than one disorder, such as a prosthodontic treatment provider, dental treatment provider, and orthodontic treatment provider, and so on. Each of the providers in communication with the first user may also utilize a communication tool for one or multiple users in addition to the first user, e.g., for receiving disorder data, for providing treatment estimates (e.g., information of one or more recommended treatments), or the like.

In some embodiments, payment methods, discounts, special offers, or the like may be evaluated by the system. One or more payment methods, including partial payments from multiple sources, may be evaluated. Payments evaluated and applied to estimates of treatment cost may include promotional prices, discount prices, partial pre-payment, partial gift payments, insurance-adjusted pricing, etc.

Methods and systems include an end-to-end digital workflow providing treatment and/or devices, where (a) a discount may be used to induce a person seeking treatment into a treatment estimation system; (b) the treatment estimation system may include a multiple-provider bidding system, which provides a list of treatment providers (e.g., doctors) that will accept the potential customer, patient, discount, etc.; (c) the customer selects, is matched with, etc. one or more treatment providers in the list; (d) the discount or other portions/sources of payment are applied toward treatment; (e) updated bills, invoices, reports, etc., are generated based on agreements associated with costs of treatment, which service providers of a treatment chain are to be responsible for discounts, etc.

Treatment may include dental treatment, cosmetic treatment, orthodontic treatment, facial treatment, Botox procedures, plastic surgery, laser treatments, etc. Devices may include appliances for affecting treatment, such as dental appliances for performing orthodontic treatment. Devices may include appliances for other applications, such as guards for sports, retainers, sleep guards, etc. Devices may include appliance subscriptions, e.g., retainers designed to be used for some period of time (e.g., a few months) and then be replaced.

In some embodiments, a discount takes the form of a gift (e.g., a physical, electronic, etc. gift card). A person can gift treatment or devices to someone else. The gift may be delivered physically or digitally, e.g., via email, via a portal associated with a distributer (e.g., a store selling gift cards), a treatment provider (e.g., an online portal or app associated with a treatment procedure or provider), or the like.

In some embodiments, an input device (mobile phone, scanner, kiosk, laptop, etc.) is used to capture photos a person seeking treatment or devices. For example, a person seeking dental treatment may capture photos of dentition, a person seeking facial cosmetic surgery may capture photos of a target facial region, etc. The photos may be sent to the treatment estimator and/or multiple provider bidding system, where doctors or other providers provide a basis to assess case complexity (and/or other factors) and provide estimates of treatment costs. The person seeking treatment, appliances, devices, etc. can then review the estimates/bids, other attributes of treatment providers who have provided information, etc.; the person seeking treatment can further select a provider.

In some embodiments, the input device is provided at a store (e.g., a store offering a discounted purchase price), an employer (e.g., as part of a company health and wellness event), etc. and is used to capture photos of a target treatment zone of a person seeking treatment or devices. The input device could be provided at an event and/or location of a promotion (concert, sporting event, youth sporting event, mall, etc.).

In some embodiments, the treatment cost is managed by an engine that allocates cost between some combination of a treatment/appliance developer or manufacturer, the treatment provider, an entity that facilitates/sells the discount (e.g., a distributer, a store, an employer, etc.), a financing entity, etc. In some implementations, the treatment provider provides a discount to, e.g., the treatment developer or the facilitating/selling entity to generate leads of potentially interested customers (so the treatment provider would be providing the discount in exchange for lead generation).

Methods and systems of the present disclosure provide technological advantages over conventional systems. Systems of the present disclosure may enable convenient communication between a user (e.g., a patient or potential patient) and a treatment provider. Systems of the present disclosure may enable a user to provide information about a disorder for treatment to a treatment provider or to multiple treatment providers in parallel. A user may provide one or more images including the disorder (e.g., pictures of dentition for orthodontic or prosthodontic treatment) to one or more treatment providers. A treatment provider or each treatment provider may evaluate a disorder of a user based on the provided information (e.g., images of dentition) and provide information of a recommended treatment (e.g., a cost and/or treatment duration estimate) to the user. Systems of the present disclosure may enable communication between a user and a number of providers, e.g., for receiving multiple estimates from multiple treatment providers that the user may then select from for pursuing further treatment. This may enable a user to receive multiple treatment estimates in parallel, to communicate with treatment providers that provided the multiple treatment estimates, and ultimately to choose a desired treatment from the provided estimates without physically visiting the treatment providers and with little expenditure of user time.

In one aspect of the present disclosure, a method includes determining, by a processing device, a plurality of treatment providers for providing a dental treatment to a user. The method further includes providing to a first user device identification of the plurality of treatment providers. The method further includes receiving image data of dentition of the user from the first user device. The method further includes receiving a first user selection of a first treatment provider of the plurality of treatment providers. The method further includes receiving a second user selection of a second treatment provider of the plurality of treatment providers from the first user device. The method further includes providing the image data to a second device associated with the first treatment provider and a third device associated with the second treatment provider. The method further includes obtaining first information of a first recommended treatment for the dentition from the second device of the first treatment provider. The method further includes obtaining second information of a second recommended treatment for the dentition from the third device of the second treatment provider. The method further includes providing the first information of the first recommended treatment and the second information of the second recommended treatment to the first user device.

In another aspect of the present disclosure, a method includes obtaining, by a processing device, identification of a plurality of treatment providers. The method further includes obtaining user input. The user input is indicative of a first disorder, a first selection of a first treatment provider of the plurality of treatment providers, and/or a second selection of a second treatment provider of the plurality of treatment providers. The method further includes providing the user input to a server device. The method further includes receiving, from the server device, first information of a first recommended treatment in association with the first disorder and the first treatment provider. The method further includes receiving second information of a second recommended treatment in association with the first disorder and the second treatment provider. The method further includes obtaining a first selection of the first recommended treatment. The method further includes providing an indication of the first recommended treatment based on the third selection of the first recommended treatment to the server device.

In another aspect of the present disclosure, a method includes obtaining, by a processing device, from a first user device, a first user request for identification of a first treatment provider. The method further includes determining a treatment provider. The method further includes providing identification of the treatment provider to the first user device. The method further includes receiving from the first user device first user data. The first user data includes data indicative of dentition of the user. The first data includes a second user request for a first treatment plan associated with the dentition and the treatment provider. The method further includes providing the first user data to a second device associated with the treatment provider. The method further includes obtaining, from the second device, information of a first recommended treatment in association with the first user data and the treatment provider. The method further includes providing to the first user device the information of the first recommended treatment.

In another aspect of the present disclosure, a method includes determining a first set of payment sources for a dental treatment. The method further includes determining one or more treatment providers for providing the dental treatment to a first user. The method further includes providing to a first user device identification of the one or more treatment providers. The method further includes obtaining information of a first recommended treatment for dentition of the first user from the first treatment provider. The information includes estimated cost of treatment incorporating the first set of payment sources. The method further includes providing the first information of the first recommended treatment to the first user device.

In another aspect of the present disclosure, a method includes determining a first set of payment sources in association with a first healthcare service. The method further includes determining one or more treatment provider for providing the healthcare service to a first user. The method further includes providing to a first user device identification of the one or more treatment providers. The method further includes obtaining first information of a first recommended treatment plan in association with the first healthcare service. The first information includes an estimated cost of treatment in accordance with the first recommended treatment plan incorporating the first set of payment sources. The method further includes providing the first information to the first user device.

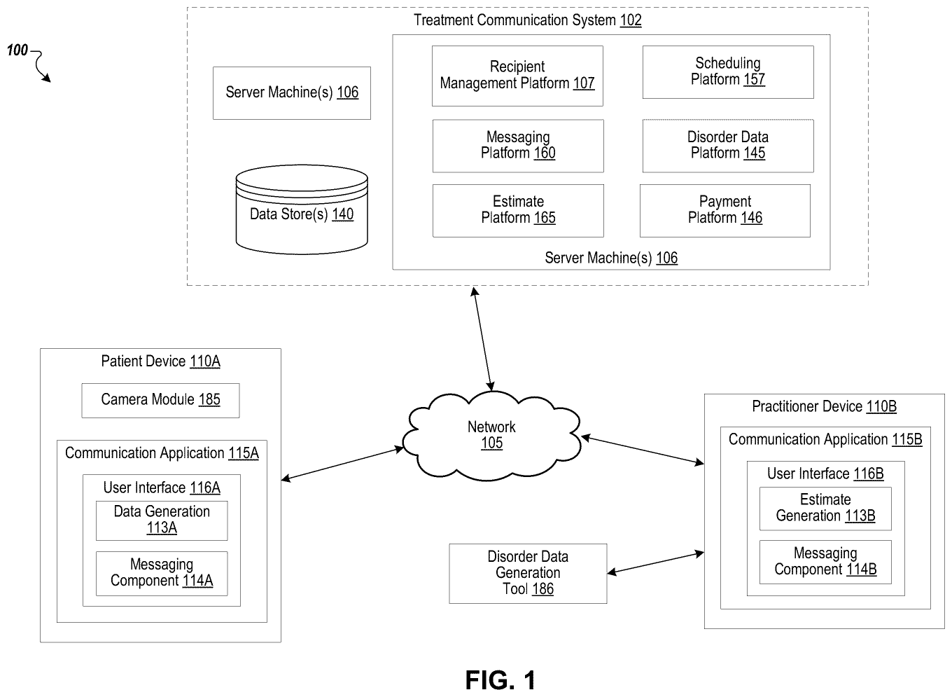

FIG. 1 illustrates an example system architecture 100 for providing tools for communication and management of aspects of treatment between a treatment provider and a user (e.g., a patient or potential patient), according to some embodiments. System architecture 100 includes a number of client devices, patient device 110A and practitioner device 110B (in some embodiments, more client devices may be included, e.g., devices 110C, 110D, etc.), one or more networks 105, and treatment communication system 102. Patient device 110A may be associated with a user, a patient, a potential patient, or the like. Practitioner device 110B may be associated with a treatment provider, such as a doctor, orthodontist, or other practitioner. Patient device 110A may include one or more tools for a user to communicate with one or more treatment providers, receive information of recommended treatment from one or more treatment providers, provide information of a disorder to one or more treatment providers, etc. Practitioner device 110B may include one or more tools for a treatment provider to communicate with one or more users (e.g., patients), for generating and/or providing estimates associated with recommended treatments, for receiving and/or reviewing data of user disorders (e.g., images of dentition), or the like.

Treatment communication system 102 includes one or more server machines 106, includes one or more data stores 140, and may include a variety of platforms targeted at performing tasks (e.g., tasks associated with content delivery). Platforms of content platform system 102 may be hosted by one or more server machines 106. Platforms of content platform system 102 may include and/or be hosted on one or more computing devices (such as a rackmount server, a router computer, a server computer, a personal computer, a mainframe computer, a laptop computer, a tablet computer, a desktop computer, etc.) and one or more data stores (e.g., hard disks, memories, and databases) and may be coupled to the one or more networks 105. In some embodiments, components of content platform system 102 (e.g., server machines 106, data stores 140, hardware associated with one or more platforms, etc.) may be directly connected to one or more networks 105. In some embodiments, one or more components of content platform system 102 may access networks 105 via another device, e.g., a hub, switch, etc. Data stores 140 may be included in one or more server machines 106, include external data storage, etc. Platforms of content platform system 102 may include recipient management platform 107, scheduling platform 157, messaging platform 160, disorder data platform 145, payment platform 146, and estimate platform 165.

The one or more networks 105 may include one or more public networks (e.g., the Internet), one or more private networks (e.g., a local area network (LAN), a wide area network (WAN), one or more wired networks (e.g., Ethernet network), one or more wireless networks (e.g., an 802.11 network), one or more cellular networks (e.g., a Long Term Evolution (LTE) network), routers, hubs, switches, server computers, and/or a combination thereof. In one implementation, some components of architecture 100 may not be directly connected to each other. In one implementation, system architecture 100 includes separate networks 105.

The one or more data stores 140 may reside in memory (e.g., random access memory), cache, drives (e.g., hard drive), flash drives, etc., and/or may be part of one or more database systems, one or more file systems, or another type of component or device capable of storing data. The one or more data stores 140 may include multiple storage components (e.g., multiple drives or multiple databases) that may also span multiple computing devices (e.g., multiple server computers). The data store may be persistent storage that is capable of storing data. A persistent storage may be a local storage unit or a remote storage unit, electronic storage units (e.g., main memory), or a similar storage unit. Persistent storage may be a monolithic device or a distributed set of devices.

Data stores 140 and server machines 106 may perform operations for storing, managing, sending, and/or receiving data associated with treatment communication system 102. Data stores 140 may store message data, communication data, disorder data (e.g., images), mapping data of recipients and/or senders of communications, treatment recommendation data, treatment estimation data, etc. Server machines 106 may manage providing data to various client devices 110 in accordance with intended data recipients, etc. Data store 140 may store patient data (e.g., images of a patient) securely and/or privately for providing to (or only to) client devices (e.g., associated with treatment providers) in accordance with user selection.

Client devices 110 (e.g., patient device 110A and practitioner device 110B) may include devices, such as televisions, smart phones, personal digital assistants, portable media players, laptop computers, electronic book readers, tablet computers, desktop computers, gaming consoles, set-top boxes, or the like. In some embodiments, a client (e.g., a user, a treatment provider, a patient, or the like) may access various tools via more than one client device (e.g., a user may be associated with a user account, may access communications via several devices such as a mobile phone, laptop, and tablet, or the like).

A client device 110 (110A, 110B, etc.) may include a communication application 115A, 115B, etc. The communication application 115A-115B may be utilized in providing communications between client devices. Communication applications 115A-115B may include purpose-built applications, web browsers accessing a website with communication functionality, or the like. Communication applications 115A-115B may be of various different designs, functionalities, or the like. For example, communication applications 115A-115B may be different for different devices, different types of client (e.g., treatment provider or potential patient), or the like.

Communication application 115A of patient device 110A may be a client side application, such as an application installed on a device of a user for inquiring about, starting, managing, etc. treatment of one or more disorders. Communication application 115A may be a purpose-built application installed on a user device. Communication application 115A may be an application association with one or more disorders, one or more treatments, one or more body parts (e.g., dentition), or the like. One example of communication application 115A is the My Invisalign® application provided by Align Technology®, Inc.

Communication application 115A may perform a number of operations to support a user (e.g., patient, potential patient, or the like). Communication application 115A may provide a doctor locator function. For example, communication application 115A may be configured to provide a list of practitioners and/or treatment providers associated with a user disorder. The list of practitioners may be based on location (e.g., of the user device or entered by the user), practice history, disorder type, etc.

Communication application 115A may provide case assessment functionality for a user (e.g., patient, potential patient). In some embodiments, communication application 115A may provide data indicative of a disorder to one or more models for case assessment. In some embodiments, the one or more models for case assessment may include one or more trained machine learning models. In some embodiments, case assessment functionality may include assigning a case complexity to a disorder based on disorder data. For example, case assessment models may assign a case complexity (e.g., via a case complexity metric) to an orthodontic disorder based on images provided to the case assessment models. In some embodiments, case complexity may be utilized in further operations. For example, a case complexity metric may be utilized in determining a set of treatment providers that may be applicable to the user, e.g., based on experience of the treatment providers. A case complexity metric may be provided to treatment providers for inclusion in generation of an estimate (e.g., estimated treatment cost, estimated treatment duration, estimated treatment plan, etc.).

Communication application 115A may include functionality for presenting treatment estimates to a user (e.g., patient, potential patient, or the like). Communication application 115A may receive (e.g., via estimate platform 165) one or more treatment estimates associated with a treatment of a user disorder (e.g., prosthodontic treatment, orthodontic treatment, or the like). Communication application 115A may present the one or more estimates to a user (e.g., via user interface (UI) 116A). The user may evaluate, approve, deny, ask questions about, or in other ways interact with the estimates.

Communication application 115A may include functionality for scheduling appointments between a user and a treatment provider. Communication application 115A may include functionality for selecting one or more potential appointment times. Communication application 115A may receive information indicative of a treatment provider's availability, and provide one or more UI elements for selecting from the presented times. Communication application 115A may receive one or more potential appointment times from a client device associated with a treatment provider, and accept user input accepting or rejecting the one or more potential appointment times. Communication application 115A may provide and/or receive treatment scheduling data in association with treatment of a user.

Communication application 115A may provide a method for a user to engage with one or more treatment payment, treatment discount, payment reduction, or other payment options with respect to a treatment. For example, a user may obtain a discount code, purchase a discount card, purchase or receive as a gift a partial payment for a healthcare service (e.g., dental or orthodontic service, though aspects of the present disclosure may be applicable to other healthcare services, such as retainers, sport guards, facial surgery, plastic surgery, botox, or other services). Via communication application 115A, a user may register or apply a payment method, such as a discount, gift card or partial payment. In some embodiments, a user may apply a payment method for their own healthcare service. In some embodiments, a user may apply a payment method for another user's healthcare service, e.g., as a gift. Communication application 115A may enable application of a giftcard or other payment method to a treatment. In some embodiments, use of a communication application 115A to register a payment method may alter further functions of system 100, e.g., treatment provider locator operations may be based or include consideration of a point of purchase or point of retrieval of the payment method (e.g., gift card). In some embodiments, a user may register a payment method via communication application 115A for another person (e.g., as a gift), and communication application 115A may include a method to alert the recipient (e.g., by sending an email indicating the gift).

Communication data associated with treatment communication system 102 may be accessed by communication application 115A. For example, communication application 115A may access one or more networks 105 (e.g., the internet) via hardware of patient device 110A to provide communication data to the user. As used herein, “communication data” may include an electronic file that can be executed or loaded using software, firmware, and/or hardware configured to present a message, disorder data, estimate data, or other communication data. In one implementation, the communication applications 115 may be applications that allow users to compose, send, and/or receive communications over a platform (e.g., scheduling platform 157, messaging platform 160, disorder data platform 145, estimate platform 165, etc.) and/or a combination of platforms and/or networks.

In some embodiments, the communication applications 115A-B may be or include social networking applications, photo sharing applications, chat applications, or a combination of such applications. The communication applications 115A-B associated with client devices 110 may render, display, present and/or play one or more communications to one or more users. For example, communication applications 115A and 115B may provide user interfaces 116A and 116B (e.g., a graphical user interface) to be displayed on endpoint devices 110A and 110B for receiving, sending, and/or viewing communication data. In some embodiments, communication applications 115A and 115B are related applications that are each associated with treatment communication system 102, and provide some overlapping features and are designed to support different types of client devices 110A and 110B respectively (e.g., mobile phone type, laptop computer type, desktop computer type, tablet computer type, TV type, and so on).

In some embodiments, communication applications 115A-B may include messaging components 114A-B. Messaging component 114A (and 114B, etc.) may provide various functionality for sending and receiving messages, e.g., between client devices. Messaging component 114A may include options to receive, view, compose, and/or send messages. Messages may be provided to one or more recipients, e.g., managed by recipient management platform 107. For example, a treatment provider may send a message directed toward a limited-time promotion to a number of different users, a user may send a treatment inquiry to multiple treatment providers, or the like.

Communication applications, client devices, or user interfaces associated with clients with different needs may include different components, enable different functionalities, or the like. User interface 116A of patient device 110A (e.g., a client device associated with a user, potential patient, user profile, or the like) may include data generation component 113A. Data generation component 113A may be utilized by a user in generating data including information of a disorder of the user, for which the user may be seeking treatment. For example, the user may be seeking orthodontic treatment, and the data generation component 113A may be utilized by a user in generating disorder data for providing to one or more treatment providers, e.g., to be used in an assessment, in generating a treatment plan, in generating a treatment cost estimate or treatment duration estimate, or the like.

Data generation component 113A may perform one or more methods for generation of disorder data. For example, data generation component 113A may perform one or more methods for generating image data of a user's dentition for use by a treatment provider in generating one or more treatment estimates. In some embodiments, image data is generated while a user wears a cheek retractor. Data generation component 113A may provide instructions to a user for disorder data generation. Data generation component 113A may provide assessment/analysis of generated disorder data. Data generation component 113A may provide instructions to modify, update, or replace disorder data that does not satisfy one or more threshold conditions. In some embodiments, data generation component 113A may perform methods including providing disorder data to one or more disorder data assessment models. In some embodiments, data generation component 113A may provide disorder data to one or more models for assessing the disorder data. In some embodiments, data generation component 113A may provide disorder data to one or more image assessment models. In some embodiments, data generation component 113A may provide disorder data to one or more trained machine learning models, statistical models, rule-based models, or the like. Data generation component 113A may prompt a user to adjust or replace disorder data based on output of the assessment models.

Patient device 110A may further include camera module 185. Camera module 185 may be utilized by a user (e.g., via user interface 116A) to generate disorder data (e.g., images of dentition of the user). Camera module 185 may be operated via communication application 115A to generate one or more images of a disorder of a user, for generating recommended treatment estimates. In some embodiments, disorder data may not be generated by patient device 110A. For example, a user may provide dentition data received from another device, such as an intraoral scanner, and this provided disorder data may be utilized in generating a treatment estimate.

In some embodiments, patient device 110A corresponds to a dental consumer/patient system such as described in U.S. Publication No. 2022/0023008 (the '008 Application), published Jan. 27, 2022, which is incorporated by reference herein in its entirety. In some embodiments, practitioner device 110B corresponds to a dental professional system as described in the '008 Application.

Communication application 115B may include functionality directed toward use by a treatment provider. Communication application 115B may be a purpose-built application for practitioners, a website for practitioners, or the like. Communication application 115B may provide access to a practitioner-side system, e.g., communication application 115B may be or include a doctor portal, such as Invisalign® Doctor Portal, provided by Align Technology®. Communication application 115B may provide functionality for a practitioner to manage one or more aspects of their practice, manage one or more treatment plans, or the like.

Communication application 115B may include estimate generation component 113B. Estimate generation component 113B may perform methods for enabling a treatment provider to provide treatment estimates to one or more users. For example, estimate generation component 113B may present disorder data to a treatment provider. Estimate generation component 113B may enable a treatment provider to request updated disorder data, to provide treatment estimates (e.g., treatment cost estimates, treatment duration estimates, estimates of treatment plans, etc.), or the like. Estimate generation component 113B may provide one or more tools for assisting a treatment provider in generating an estimate. For example, estimate generation component 113B may evaluate (e.g., via one or more models, such as trained machine learning models) disorder data and provide guidance on predicted treatment. Estimate generation component 113B may provide a complexity metric for a patient or potential patient. Estimate generation component 113B may provide recommendations of disorder types, disorder severity, potentially required treatments (e.g., types of orthodontic equipment that may be included in a treatment plan), or the like.

Communication application 115B may provide information regarding payment methods with respect to a healthcare service. For example, alerts regarding application of discounts, partial payments, insurance payments, or the like may be provided to a practitioner via communication application 115B. In one example, a practitioner may provide a discount on services, e.g., in exchange for marketing or lead generation. Communication application 115B may obtain data (e.g., via user entry of a discount code by communication application 115A) indicative of the discount, instructing the practitioner of partial payment, of one or more payment methods, or the like.

Communication application 115B may provide additional functionality to a treatment provider. Communication application 115B may provide functionality to manage treatments, generate and/or manage treatment plans, generate and/or manage treatment estimates, and the like for treatment providers. Communication application 115B may provide functionality to review active cases, potential cases, submitted cases, etc. Communication application 115B may provide functionality for tracking cases in progress.

In some embodiments, a treatment provider may generate disorder information via practitioner device 110B. For example, practitioner device 110B may be coupled to a disorder data generation tool 186. Disorder data generation tool 186 may be any device, communicatively coupled to practitioner device 110B (either directly, as shown, or via one or more networks 105), that may collect or generate user disorder data. As examples, disorder data generation tool 186 may be a camera, a computed tomography device, an X-ray device, an intraoral scanning device, or another device for generating, collecting, and/or measuring disorder data.

In some implementations, applications 115A-B are web browsers that can access, retrieve, present, and/or navigate content (e.g., web pages such as Hyper Text Markup Language (HTML) pages, digital media items, etc.) and can include messaging components, data generation components, estimate generation components, or other components that may be related to treatment communication system 102. Client interaction with communication application 115B may be via user interface 116 (e.g., a web page associated with treatment communication) provided by treatment communication system 102. Alternatively, application 115 is not a web browser and is a stand-alone application (e.g., mobile application, desktop application, gaming console application, television application, etc.), that is downloaded from a or pre-installed on the client device 110. The stand-alone application 115 can provide user interface 116 including the various components. It will be understood that components and elements associated with practitioner device 110B (e.g., communication application 115B, user interface 116B, etc.) may have similar features as those described as exemplary features of components and elements associated with patient device 110A.

In some embodiments, client devices 110 may present a different user experience to a user. For example, patient device 110A may be a first screen device with features targeted toward use by a patient or potential patient. For example, patient device 110A may be a mobile device, such as a smartphone, tablet or laptop, including touchscreen navigation and/or a virtual keyboard, a desktop computer including a cursor control device (e.g., mouse) and keyboard, etc., owned by or associated with a user profile of a patient or potential patient. Practitioner device 110B may be a second screen device (including a device coupled to a screen, such as a gaming console) with features targeting use by a treatment provider, e.g., practitioner device 110B may be a device owned by or associated with the treatment provider.

In some embodiments, communication applications 115 installed on client devices 110 may be associated with a user account, e.g., a user may be signed in to an account on the client device 110. In some embodiments, multiple client devices 110 may be associated with the same client account. In some embodiments, generating a communication link between client devices (e.g., via treatment communication system 102, in association with recipient management platform 107, or the like) may be performed responsive to the client devices being associated with the same user account.

In some embodiments, client devices 110 may include one or more data stores. Data stores may include commands (e.g., instructions, which cause operations when executed by a processing device) to render a UI (e.g., user interfaces 116). The instructions may include commands to render various components for use in communication regarding treatment, such as messaging components, data generation components, estimate generation components, etc.

In some embodiments, the one or more server machines 106 may include computing devices such as rackmount servers, router computers, server computers, personal computers, mainframe computers, laptop computers, tablet computers, desktop computers, etc., and may be coupled to one or more networks 105. Server machines 106 may be independent devices or part of any of the platforms (e.g., disorder data platform 145, messaging platform 160, etc.). In embodiments, recipient management platform 107, messaging platform 160, estimate platform 165, scheduling platform 157 and/or disorder platform 145 include software (e.g., optionally implemented as software as a service SaaS) and/or supporting infrastructure (e.g., hardware, firmware, virtual machines, etc.) for performing various services and/or operations.

Recipient management platform 107 may manage connections between client devices, users, user accounts, or the like. For example, recipient management platform 107 may manage recipients of messages, data, images, appointment data, or other information. Recipient management platform 107 may manage one-to-one connections, one-to-many connections, many-to-one connections, many-to-many connections, etc. such as providing data indicative of a disorder from a user device to a number of devices associated with various treatment providers associated with the disorder.

Scheduling platform 157 may manage appointment scheduling operations in connection with treatment communication system 102. For example, responsive to one or more actions via a client device (e.g., generation of a treatment estimate, selection of a treatment provider, or the like), scheduling of an appointment in association with the treatment between a user and a treatment provider may be initiated. In some embodiments, one or more tools provided in connection with treatment communication system 102 (e.g., communication applications 115) may include scheduling components for scheduling appointments between a user and a treatment provider.

Messaging platform 160 may manage messaging between client devices in association with treatment communication system 102. For example, messages requesting or providing additional estimate information, additional disorder information, updated estimates, appointment updates, or the like may be provided via communication applications 115.

Disorder data platform 145 may manage disorder data in association with treatment communication system 102. For example, disorder data platform 145 may manage data privacy, including managing secure storage, receiving, and sending of disorder data (e.g., images of users).

Estimate platform 165 may manage estimate data in association with treatment communication system 102. Estimate platform 165 may manage privacy, receipt, delivery, updating, etc., associated with treatment estimates.

Payment platform 146 may manage one or more aspects of integrating various payment methods for healthcare services, such as dental or orthodontic care, in connection with other components of system 100. For example, payment platform 146 may obtain input from communication application 115A, integrate multiple payment sources (e.g., partial payment, discount, prepayment, insurance payment, gift card, etc.), provide an indication of cost and/or patient charge to communication application 115B, provide and/or record a history of payments, indicate to a practitioner how much should be charged, how much to adjust a standard charge by, or the like in association with a target patient.

Further to the descriptions above, a user may be provided with controls allowing the user to make an election as to both if and when systems, programs, or features described herein may enable collection of user information (e.g., information about a user's social network, social actions, or activities, profession, a user's preferences, or a user's current location), and if the user is sent content or communications from a server. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user.

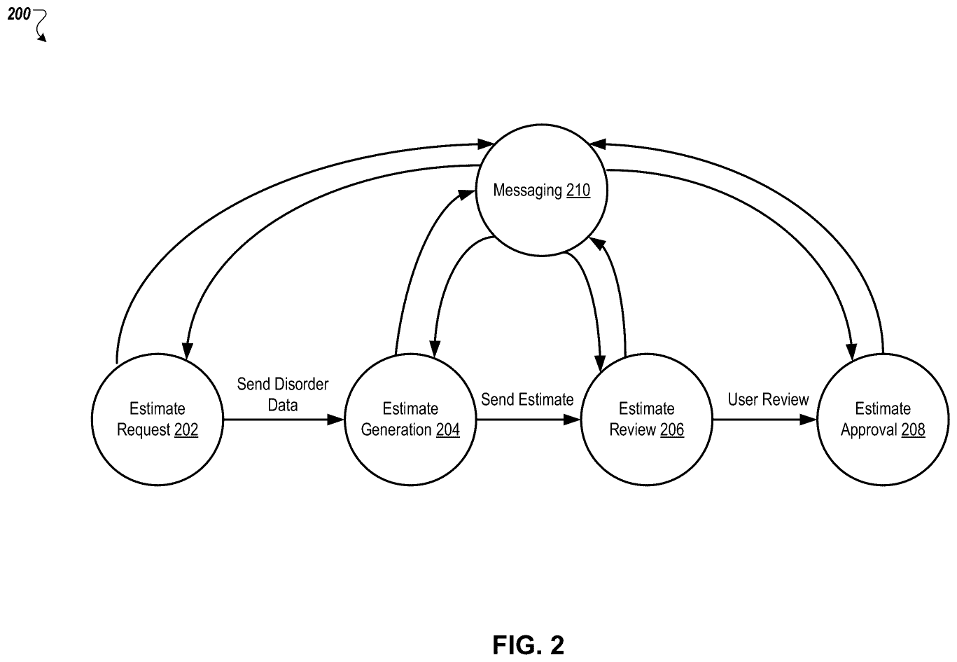

FIG. 2 is a diagram of a flow 200 for providing treatment estimates to a user (e.g., via treatment communication system 102 of FIG. 1), according to some embodiments. Data may be generated, transmitted, and/or received by one or more client devices, such as a patient device and a practitioner device. Interaction of the user at a first device may be performed via an application, such as the My Invisalign® application provided by Align Technology®, Inc. Interaction of the practitioner at a second device may be performed via another application, such as a web browser providing an Invisalign® Doctor Portal, provided by Align Technology®.

At block 202, an estimate request is generated. Generation of an estimate request may be performed by a user device, e.g., a patient device. Generation of an estimate request may include generating disorder data. Generation of an estimate request may include generating one or more images including information of a disorder for treatment. Generation of an estimate request may include generating images of a user's dentition. Generation of an estimate request may include providing instructions to a user guiding generation of disorder data. For example, generation of an estimate request may include providing instructions for a user to take one or more pictures of the user's teeth, such as instructions regarding areas to include, view angles, other contributing actions such as lip retraction, etc. Generation of an estimate request may include a user sending a request for one or more treatment provider recommendations, and a user device receiving a set of one or more treatment providers responsive to the request. Generation of an estimate request may include providing information related to one or more payment sources, e.g., a gift card purchased by or for the user, a discount related to a promotional event attended by the user, or the like.

At block 204, an estimate is generated. Estimate generation operations may be performed by a client device, practitioner device, doctor device, or the like. Estimate generation may include treatment provider input based on disorder data. Estimate generation may include generation of an estimated cost of treatment. Estimate generation may include generation of an estimated duration of treatment. Estimate generation may include generation of an estimated treatment plan, e.g., including techniques, materials, devices, or the like that may be used in treatment of a disorder. In some embodiments, the estimate generated may include or be responsive to one or more payment sources, e.g., gift cards, prepayment, discounts, projected insurance coverage, or the like. An alert including the estimate generation may include an indication of one or more payment sources, e.g., may indicate an estimated cost of treatment, an estimated amount of that cost mitigated by one or more factors, indicate an estimated out of pocket cost, or the like.

At block 206, estimate review is performed. Estimate review operations may be performed by a user device. Estimate review may be performed by a user. Estimate review may include presenting one or more treatment estimates to a user. Estimate review may include review of estimates provided by multiple treatment providers. Estimate review may include evaluation of a preferred treatment based on treatment estimates provided by multiple treatment providers.

At block 208, estimate approval is performed, which may include approval of a proposed treatment plan, user selection of one treatment from a number of treatment presented to the user, or the like.

Messaging 210 may include operations for communication between one or more users and one or more treatment providers outside the estimate providing and review flow. Messages may be processed to or from a treatment provider or a user at various stages of estimate flow. For example, messages may be provided by a user requesting some additional information from a treatment provider before requesting an estimate, a message may be sent by a treatment provider requesting updated disorder data, a message may be sent by a user requesting an updated estimate, or the like.

A backend server (e.g., server machine 106 of FIG. 1) may be utilized to manage various states of communication. For example, a server may transmit messages, estimates, disorder data, and the like between one or more user devices and one or more treatment provider devices. A server may manage data delivery, data privacy, user settings, and the like. In some embodiments, one or more states of flow 200 may trigger a device to provide a notification. For example, upon receiving a message, disorder data, an estimate, or the like, a device may provide a notification to alert a user or treatment provider.

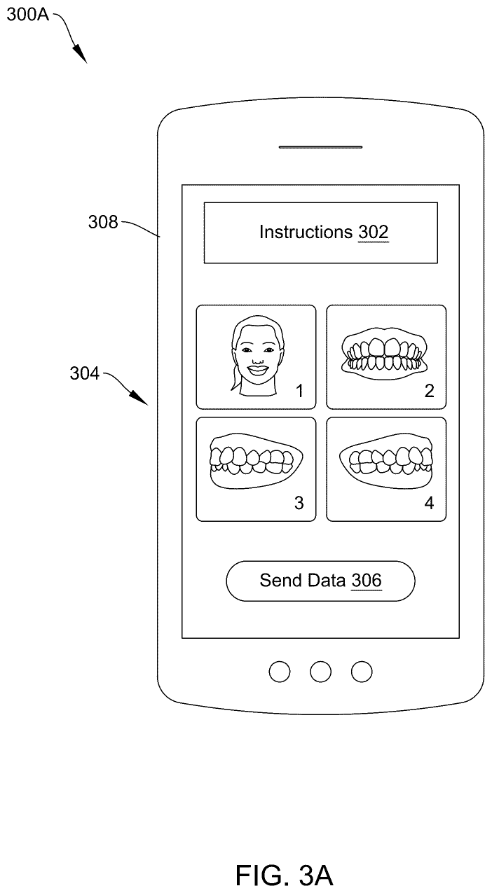

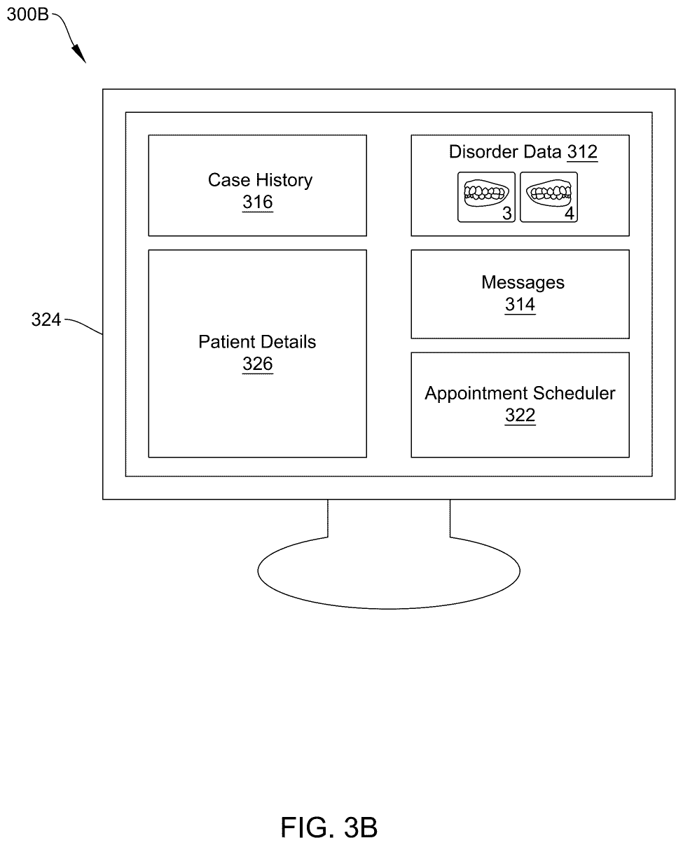

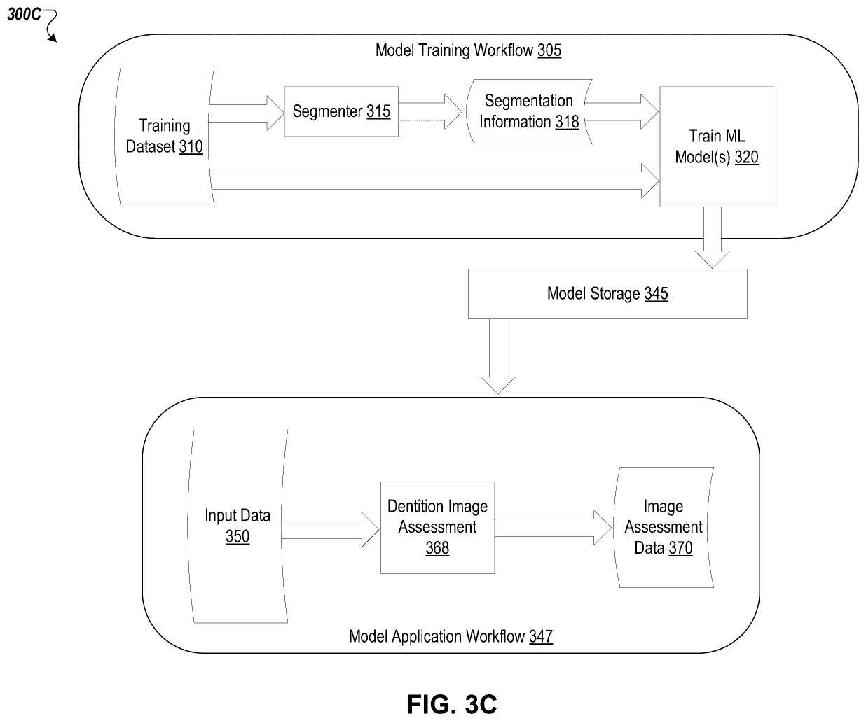

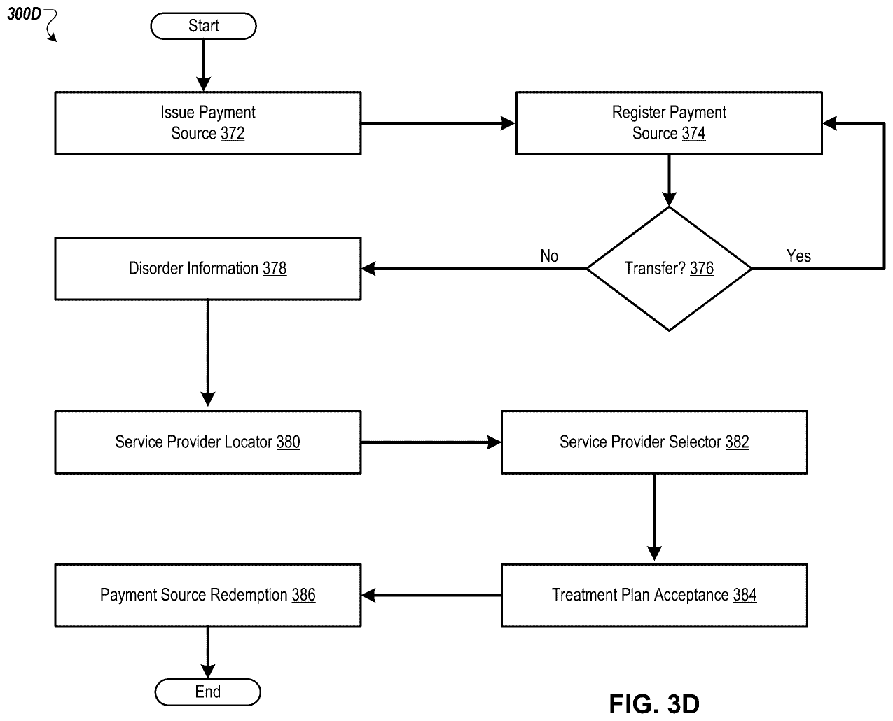

FIG. 3A depicts an example graphical user interface (GUI) 300A of a user device 308 for providing disorder data, according to some embodiments. GUI 300A includes user interface (UI) elements for interaction by a user for generation of disorder data.

In some embodiments, disorder data may include a number of images of a patient. Disorder data may include a number of images including the disorder, and/or related or nearby regions. Disorder data may include a number of images of a user's dentition, e.g., for orthodontic or prosthodontic disorders. GUI 300A may be executed by an application installed on user device 308, such as a purpose-built application, a web browser accessing a website associated with treatment management and/or estimate review, or the like.

In some embodiments, instead or in addition to generating disorder data, a user may supply disorder data generated separately. For example, a user may receive additional data indicative of a user disorder from a medical service, imaging device, or the like, and provide the data via user device 308 to one or more treatment providers. For example, a user may receive x-ray data, computed tomography data, three-dimensional scan data, or the like (for example, from a third party or a different treatment provider), and may provide the disorder data to one or more treatment providers via user device 308.

GUI 300A may provide one or more elements to guide a user to generate disorder data. For example, a written instructions element, instructions 302, may be provided to instruct a user in generating disorder data that may be utilized by a treatment provider in generating a treatment estimate. Further, visual guides 304 may be provided. Visual guides 304 may include visual instructions for generation of disorder data, for example, sample images or drawings demonstrating a target image. In some embodiments, visual guides 304 may be interactable elements, which cause a camera module to operate for generation of disorder data. In some embodiments, images presented in visual guides 304 may be replaced as disorder data is generated, e.g., with images of the user's dentition.

In some embodiments, disorder data generation may be augmented by one or more image assessment models. As part of the disorder data generation process, one or more images may be provided to image assessment models. The image assessment models may include trained machine learning models, rule-based models, statistical models, or the like. The image assessment models may be applied during the image capturing process, soon after capturing images, or the like. The image assessment models may help a user to generate disorder data that is able to be evaluated by a treatment provider for generation of a treatment estimate. The image assessment models may determine whether one or more image deficiencies are present in the image data. The image assessment models may be rule-based, e.g., may measure brightness, contrast, sharpness, or another metric of interest and provide guidance to a user to improve the metric values. The image assessment models may include trained machine learning models, that may be trained to recognize acceptable disorder data, trained to identify deficiencies in disorder data, or the like. More discussion of machine learning image assessment may be found in connection of FIG. 3C. In some embodiments, one or more models (e.g., machine learning models) may perform case evaluation. For example, based on dentition images, a model may generate a predicted complexity metric value for a disorder.

In some embodiments, GUI 300A may further provide a send data UI element 306. Sending disorder data may include providing disorder data (e.g., images of dentition) to one or more treatment providers. Sending disorder data may include providing disorder data to a list of treatment providers selected by a user.

FIG. 3B depicts a GUI 300B for enabling a treatment provider to provide a treatment estimate, according to some embodiments. GUI 300B may be provided via practitioner device 324. GUI 300B may be provided as part of an application, a doctor portal, a website, or the like. GUI 300B may present various information associated with a patient, a user, a case, a treatment, or the like. In some embodiments, the application may present one or more UI elements for a treatment provider to select a particular case (e.g., patient) from a set of patients that may be assessed via the application.

Upon selection of a case, information about the case may be presented to a treatment provider. Patient details 326 and case history 316 may be utilized by the treatment provider in reviewing the case, updating treatment plans, determining future treatment steps, and the like.

Disorder data 312 may be presented to a treatment provider. The disorder data 312 may include images including one or more user disorders for evaluation by the treatment provider. A UI element may be included for further review of disorder data (e.g., to enlarge images). A UI element may be included to provide estimates to the user, e.g., a “send estimates” element. A UI element may be included to perform additional estimate generation operations. For example, disorder data may be provided to one or more models for assisting a treatment provider in generating an estimate. The one or more models may estimate a case complexity, predict a treatment plan, or the like. In some embodiments, a treatment provider may provide some analysis to a model as additional input to the disorder data, or instead of the disorder data. For example, a treatment provider may provide an estimated case complexity metric value, one or more disorders, one or more severity metric values, one or more treatment techniques, or the like to a model, which may assist in generating a treatment estimate based on the treatment provider input and/or the disorder data.

Messages 314 may include communications between a treatment provider and a user. History of messages transmitted and received may be displayed. A field for composing and/or sending a new message may be included.

Appointment scheduler 322 may include UI elements for scheduling appointments with a user. Appointment scheduler 322 may include functionality to accept an appointment proposed by a user. Appointment scheduler 322 may include one or more UI elements for proposing appointment times to a user. Appointment scheduler 322 may include a history of past appointments, indications of upcoming appointments, and the like.

In some embodiments, GUI 300B may provide information indicative of one or more payment sources in association with a patient. For example, a point of sale of a discount or other offer, a marketing offer or event attended by the patient, an adjusted price to charge the patient based on payment sources, or the like may be indicated via GUI 300B.

FIG. 3C illustrates workflows 300C for training and implementing one or more machine learning models for performing operations associated with treatment estimate generation, in accordance with some embodiments. The illustrated workflows include a model training workflow 305 and a model application workflow 347. The model training workflow 305 is to train one or more machine learning models (e.g., deep learning models, generative models, etc.) to perform one or more data segmentation tasks and/or data generation tasks (e.g., for images of smiling persons showing their teeth, intraoral images, images of an impression taken of a dental arch, images of physical or digital three-dimensional dental models, etc.). The model application workflow 347 is to apply the one or more trained machine learning models to generate image assessment data based on the input data 350. A similar workflow may be utilized for different data types, such as a machine learning model that is configured to predict a case complexity based on images of dentition, a model that is configured to classify one or more disorders based on disorder data, a model that is configured to generate a predicted treatment plan based on disorder data, or the like.

Training of a machine learning model such as a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

The model training workflow 305 and the model application workflow 347 may be performed by processing logic, executed by a processor of a computing device. Workflows 305 and 347 may be implemented, for example, by one or more devices depicted in FIG. 1, such as server machine 106, patient device 110A, practitioner device 110B, etc. These methods and/or operations may be implemented by one or more machine learning modules executed on processing devices of devices depicted in FIG. 1.

For the model training workflow 305, a training dataset 310 containing hundreds, thousands, tens of thousands, hundreds of thousands or more examples of input data may be provided. The properties of the input data will correspond to the intended use of the machine learning model(s). For example, a machine learning model for assessing disorder images may be trained. Training the machine learning model for assessing disorder images may include providing a training dataset 310 of images of related to the target disorder images, such as images of dentition.

Training dataset 310 may include various types of data, e.g., various representations of dental arches. Training dataset 310 may include different types of data for different intended uses of machine learning models. Training dataset 310 may include three-dimensional scan data for a machine learning model configured to accept three-dimensional scan data as input. Training dataset 310 may include two-dimensional images including jaw pair information for training a machine learning model to accept two-dimensional images as input. Training dataset 310 may include one or more user classifications, such as treatment provider estimates of complexity, recommendations of treatment tools, or the like, for a model configured to receive this data as input. Training dataset 310 may include computed tomography (e.g., cone-beam computed tomography), x-ray, or other types of data for training a machine learning model to accept these data types as input. Training dataset 310 may include additional information, such as contextual information, metadata, etc. Training dataset 310 may include positional data associated with one or more teeth, orientation data, spatial relationship data, etc. Training dataset 310 may include data that may be associated with predictions of any type of dental issue that may affect treatment of a disorder. For example, results of orthodontic, restorative, or ortho-restorative treatments may be predicted, ongoing tooth wear, tooth decay, changes to teeth due to jaw development (e.g., as a child or adolescent grows), etc.

Training dataset 310 may reflect the intended use of the machine learning model. For training of a machine learning model to assess image quality for generating a treatment estimate, training dataset 310 may include images of a subject (e.g., target region, dentition, etc.) and image classifications, such as classification of whether the image is acceptable, classification of reasons an image does not meet target standards (e.g., out of focus, subject too far away, image too dark, etc.). For training of a machine learning model to predict complexity of a case, training dataset 310 may include disorder data (e.g., images, three-dimensional scans, x-rays, metadata or user-provided classifications, etc.) and assigned case complexity. For training of a machine learning model to predict recommended treatment, disorder data (e.g., dentition images) and associated treatment plans may be included in training dataset 310.

In some embodiments, some or all of the training dataset 310 may be segmented. For example, a model may be trained to separate teeth from an image, classify disorders in a more granular manner (e.g., a first tooth requires some positional treatment, a second tooth requires some rotational treatment, etc.). The segmenter 315 may separate portions of dental arch data for training of a machine learning model. For example, individual teeth of dental arch data may be utilized as training input for a model configured perform operations for generating treatment estimates. Individual teeth, groups or sets of teeth, arches, jaws, or the like may be segmented from jaw pair or dental arch data to train a model. Segmenting data may enable higher accuracy of complexity predictions, treatment predictions, etc.

Data of the training dataset 310 may be processed by segmenter 315 that segments the data of training dataset 310 (e.g., jaw pair data) into multiple different features. The segmenter may then output segmentation information 318. The segmenter 315 may itself be a machine learning model, e.g., a machine learning model configured to identify individual teeth or target groups of teeth from dental arch data. Segmenter 315 may perform image processing and/or computer vision techniques or operations to extract segmentation information 318 from data of training dataset 310. In some embodiments, segmenter 315 may not include a machine learning model. In some embodiments, training dataset 310 may not be provided to segmenter 315, e.g., training dataset 310 may be provided to train ML models without segmentation.

In some embodiments, various other pre-processing operations (e.g., in addition to or instead of segmentation) may also be performed before providing input (e.g., training input or inference input) to the machine learning model. Other pre-processing operations may share one or more features with segmenter 315 and/or segmentation information 318, e.g., location in the model training workflow 305. Pre-processing operations may include mesh closing, artifact removal, outlier removal, smoothing, registration (bringing various meshes into the same topology), or other pre-processing that may improve performance of the machine learning models.

Data from training dataset 310 may be provided to train one or more machine learning models at block 320. Training a machine learning model may include first initializing the machine learning model. The machine learning model that is initialized may be a deep learning model such as an artificial neural network. An optimization algorithm, such as back propagation and gradient descent may be utilized in determining parameters of the machine learning model based on processing of data from training dataset 310.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many inputs in the training dataset yields a network that can produce correct output when presented with inputs that are different than the ones present in the training dataset. In high-dimensional settings, such as large images, this generalization is achieved when a sufficiently large and diverse training dataset is made available.

An artificial neural network includes an input layer that consists of values in a data point (e.g., intensity values and/or height values of pixels in a height map). The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer.

Processing logic adjusts weights of one or more nodes in the machine learning model(s) based on an error term. The error term may be based upon a difference between output of the machine learning model and target output provided as part of training dataset 310. The error term may be based on a difference between output of the machine learning model and training input, such as in the case of a classifier, a difference between the target output classification (e.g., classifying that an image that is too bright) and the classification assigned by the machine learning model. Based on this error, the artificial neural networks adjust one or more of their parameters for one or more of their nodes (the weights for one or more inputs of a node). Parameters may be updated in a back propagation manner, such that nodes at a highest layer are updated first, followed by nodes at a next layer, and so on. An artificial neural network contains multiple layers of “neurons”, where each layer receives as input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

In some embodiments, portions of available training data (e.g., training dataset 310) may be utilized for different operations associated with generating a usable machine learning model. Portions of training dataset 310 may be separated for performing different operations associated with generating a trained machine learning model. Portions of training dataset 310 may be separated for use in training, validating, and testing of machine learning models. For example, 60% of training dataset 310 may be utilized for training, 20% may be utilized for validating, and 20% may be utilized for testing.

In some embodiments, the machine learning model may be trained based on the training portion of training dataset 310. Training the machine learning model may include determining values of one or more parameters as described above to enable a desired output related to an input provided to the model. One or more machine learning models may be trained, e.g., based on different portions of the training data. The machine learning models may then be validated, using the validating portion of the training dataset 310. Validation may include providing data of the validation set to the trained machine learning models and determining an accuracy of the models based on the validation set. Machine learning models that do not meet a target accuracy may be discarded. In some embodiments, only one machine learning model with the highest validation accuracy may be retained, or a target number of machine learning models may be retained. Machine learning models retained through validation may further be tested using the testing portion of training dataset 310. Machine learning models that provide a target level of accuracy in training operations may be retained and utilized for future operations. At any point (e.g., validation, testing), if the number of models that satisfy a target accuracy condition does not satisfy a target number of models, training may be performed again to generate more models for validation and testing.

Once one or more trained machine learning models are generated, they may be stored in model storage 345, and utilized for generating predictive data associated with treatment estimates, such as assessing image quality for estimate generation, predicting case complexity, predicting treatment plans, etc.

In some embodiments, model application workflow 347 includes utilizing the one or more machine learning models trained at block 320. Machine learning models may be implemented as separate machine learning models or a single combined (e.g., hierarchical or ensemble) machine learning model in embodiments.

Processing logic that applies model application workflow 347 may further execute a user interface, such as a graphical user interface. A user may select one or more options using the user interface. Options may include selecting which of the trained machine learning models to use, selecting which of the operations the trained machine learning models are configured to perform to execute, customizing input and/or output of the machine learning models, or the like. For example, a user may only be interested in output of a subsection of a jaw pair (e.g., only front teeth for a smile reconstruction, only an upper jaw for treatment of an upper jaw malocclusion, etc.) and may request only output related to the target subsection. The user interface may additionally provide options that enable a user to select values of one or more properties. For example, a training plan which focuses on particular movements of target teeth may be selected. The user interface may additionally provide options for selecting an input imaging modality (e.g., first type of dental arch data such as intraoral scans, 3D models, 2D images, etc.) and/or an output format.

Input data 350 is provided to a machine learning model trained in block 320. The input data 350 may correspond to at least a portion or training dataset 310, e.g., be the same type of data, data collected by the same measurement technique, data that resembles data of training dataset 310, or the like. Input data 350 may include dental arch data, dental arch measurement data, dental arch latent space representation data, disorder data, images including a user's dentition, etc. Input data 350 may further include ancillary information, metadata, labeling data, etc. For example, data indicative of a location, orientation, or identity of a tooth or patient, data indicative of a relationship (e.g., a spatial relationship) between two teeth, a tooth and jaw, two dental arches, or the like, or other data my be included in input data 350 (and training dataset 310).

In some embodiments, input data may be preprocessed. For example, preprocessing operations performed on the training dataset 310 may be repeated for at least a portion of input data 350. Input data 350 may include segmented data, data with anomalies or outliers removed, data with manipulated mesh data, or the like.

Input data is provided to dentition image assessment 368. Dentition image assessment 368 generates image assessment data 370 based on the input data 350. In some embodiments, dentition image assessment 368 includes a single trained machine learning model. In some embodiments, dentition image assessment 368 includes a combination of multiple trained machine learning models. For example, a first trained machine learning model may determine whether an image satisfies a first quality metric (e.g., image contrast) and a second machine learning model may determine whether an image satisfies a second quality metric (e.g., image sharpness). In another example, a first trained machine learning model may determine whether an image is indicative of a first type of disorder (e.g., a disorder associated with a first treatment type), and a second machine learning model may determine whether an image is indicative of a second type of disorder. In some embodiments, dentition image assessment 368 may include a combination of machine learning models and other models. For example, a combination of machine learning models and numerical optimization models may be included in dentition image assessment 368. A combination of machine learning and statistical models may be included, e.g., to check whether the image includes features that would cause the image to be anomalous compared to a pool of images. A combination of machine learning models and rule-based models may be included, e.g., some aspects of assessing an image may be performed by rule-based checking (e.g., average image brightness). In some embodiments, all operations of dentition image assessment 368 may be performed by models that are not machine learning models. In some embodiments, a corrective action may be performed based on the image assessment data 370. The corrective action may include providing an alert to a user, designing a treatment plan, updating a treatment plan, assigning a complexity value, presenting an alert to adjust a dentition image, or the like.

FIG. 3D is a block diagram of a flow 300D for obtaining a treatment estimate, including multiple payment sources, according to some embodiments. One or more operations included in flow 300D may be performed by a user device, including a patient or practitioner device; a server related to a healthcare service provider, vendor, appliance manufacturer, or the like; an entity for supporting healthcare services; or the like. At block 372, a payment source is issued. The payment source may be a gift card purchased at a retailer, in cooperation with one or more healthcare service providers. The payment source may be a promotion, discount, employment benefit, or other source that may be obtained to partially or fully fund a healthcare service. In some embodiments, a discount may be included in a payment source, e.g., a gift card may be purchased that reduced a cost of treatment by more than the cost of the gift card (e.g., as a special promotion, as a marketing tool, etc.). In some embodiments, a discount code or gift card may be limited to one per person or customer. A unique customer log-in, unique redemption code, or the like may be utilized to ensure a threshold number of promotions, payment sources, or the like is not exceeded. In some embodiments, a payment source may be secured (e.g., a gift card purchased) in person, online, or via another method. In some embodiments, a partial payment package may be purchased, which may include physical items as well as a gift code, gift card, gift voucher, or the like related to a healthcare service. In some embodiments, a package including physical items may be shipped to a recipient, e.g., along with a gift card, gift code, or the like.

At block 374, the payment source is registered. In some embodiments, an electronic gift card, voucher code, discount code, or another delivery method is provided to a user. The user may utilize an electronic device (e.g., patient device 110A of FIG. 1) to register the payment source. The user may utilize a communication application including a web portal, downloadable app, or another means for accessing a registration for the payment device. In some embodiments, a code may be entered, a visual code (e.g., bar code, QR code, or the like) may be scanned, or a payment source identified in another way to register the payment source. In some embodiments, registering the payment source may include additional steps, such as accepting terms and conditions.

Flow splits at block 376, dependent upon whether the initial registration of the payment source is by a user to receive treatment, or another user (e.g., as a gift). In some embodiments, the communication application (e.g., app, web portal, etc.) used to register the payment source may provide an option to gift the payment source. In some cases, a gift giver may provide a code or card directly to a gift recipient, and the flow may be adjusted accordingly. If the user determines to transfer the partial payment to another user, the communication platform may enable providing a code or gift card to the second user, e.g., by entering their email address. Flow may then return to block 374, and the second user may register the partial payment for their own use. If the payment source is not to be transferred, flow continues to block 378.

At block 378, disorder information is collected, provided, generated, etc. For example, one or more questions, fields, or the like may be responded to by a user with respect to a healthcare disorder, desired healthcare services, or the like. Further operations may be based on such responses, e.g., a treatment provider locator may provide locations of treatment providers supplying the target healthcare service, treatment providers that provide a first healthcare service (e.g., supplying of replacement or tooth implants before orthodontics) of a set of healthcare services, treatment providers with multiple specialties to treat multiple of a patient's disorders, treatment providers that share facilities, offices, are nearby, or the like other healthcare services that may be of interest to the user, etc. In some embodiments, collecting disorder information may include providing images, videos, description, or the like of one or more healthcare disorders for generation of a treatment plan, generation of treatment cost estimates, or the like.

At block 380, service provider locator operations are performed. A search for healthcare service providers may be performed. The search may be initiated by a central server, initiated by a user device, etc. The search may be based on a location of a user device. The search may be based on a location entered by a user, e.g., target zip code. The search may be based on a point of sale or point of acquiring of the payment source (e.g., location of a store at which a gift card was purchased).

At block 382, a service provider selector may be presented to a user. In some embodiments, the service provider selector may include treatment cost or duration estimates. The service provider selector may include other estimates related to treatment, e.g., based on disorder information associated with block 378. The service provider selector may include or incorporate payment sources, such as discounts or partial payments applied or to be applied to the healthcare service. In some embodiments, whether or not a healthcare service provider accepts a target payment source (e.g., whether a practitioner is participating in a target promotion) may be indicated on the service provider selector.

Upon selection of a service provider, flow continues to treatment plan acceptance at block 384. Treatment plan acceptance may be performed after additional actions are taken, e.g., more in depth information is gathered by the selected service provider to generate a more detailed or complete treatment plan. In some embodiments, the additional information may be at least partially gathered at an in-person appointment, e.g., for the healthcare provider to examine the potential patient. In some embodiments, a in-person appointment (e.g., an initial in-person consultation) may include performing an instrumental examination, e.g., an oral scan utilizing dental scanning devices. In some embodiments, methods may be provided for a user to switch to a different provider, e.g., if the user wishes to change providers after an initial in-person appointment, if the estimated cost of service does not match the proposed treatment plan, or the like. Acceptance of the treatment plan may include providing a detailed treatment cost, including various payment sources (e.g., discounts, insurance payments, financing options, out of pocket costs, etc.). After accepting a treatment plan, flow continues to block 386, with redemption of the payment source, which may include flagging a unique payment source (e.g., a gift card) as expended, and the one or more target healthcare services may proceed. In some embodiments, at any point, a user may decide not to proceed with the healthcare services, and in some embodiments a return of the partial payment may be enabled. In some embodiments, an unused payment source (e.g., an unused gift card) may be automatically refunded after a period of time (e.g., two years). In some embodiments, a server may generate one or more reminders (e.g., every six months) that partial payment has been secured, encouraging a user to seek and/or proceed with treatment, healthcare services, orthodontics, or the like.

In some embodiments, variations or permutations of the operations discussed in connection with FIG. 3D may be performed. Various blocks may not be performed, may be performed by one or more different devices, may be performed in different orders, etc. Operations described in association with one block may be performed as part of another block, multiple blocks may be combined, or other changes to the flow may be enacted, and be within the scope of this disclosure.

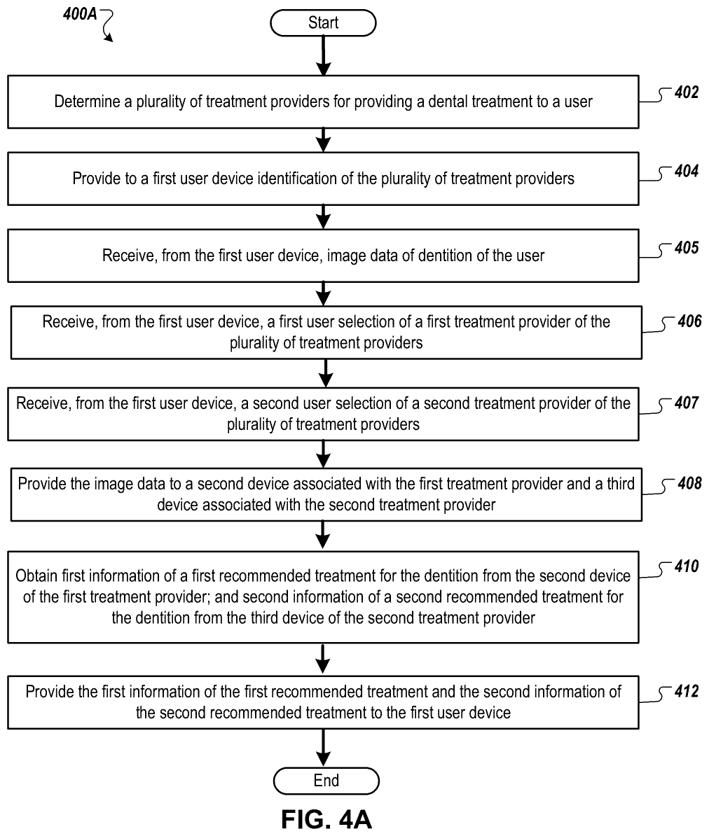

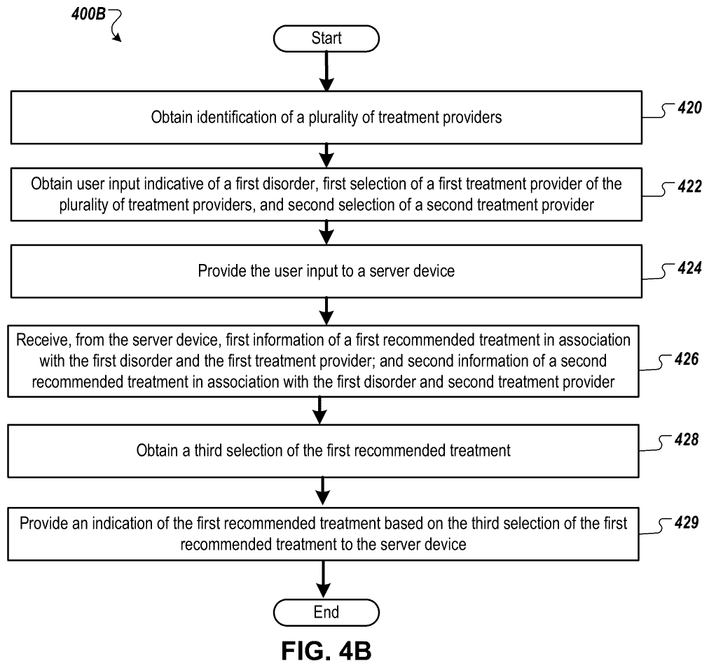

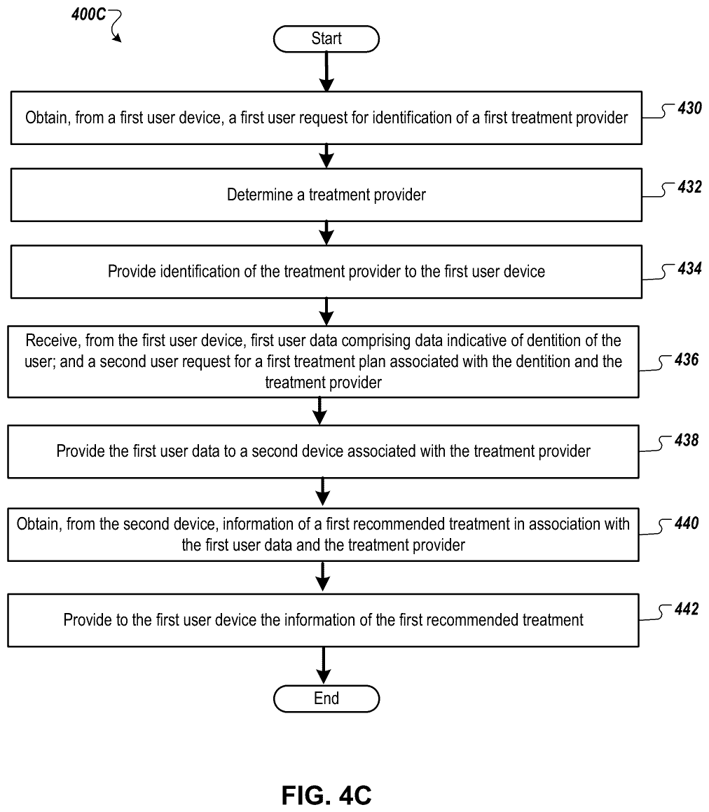

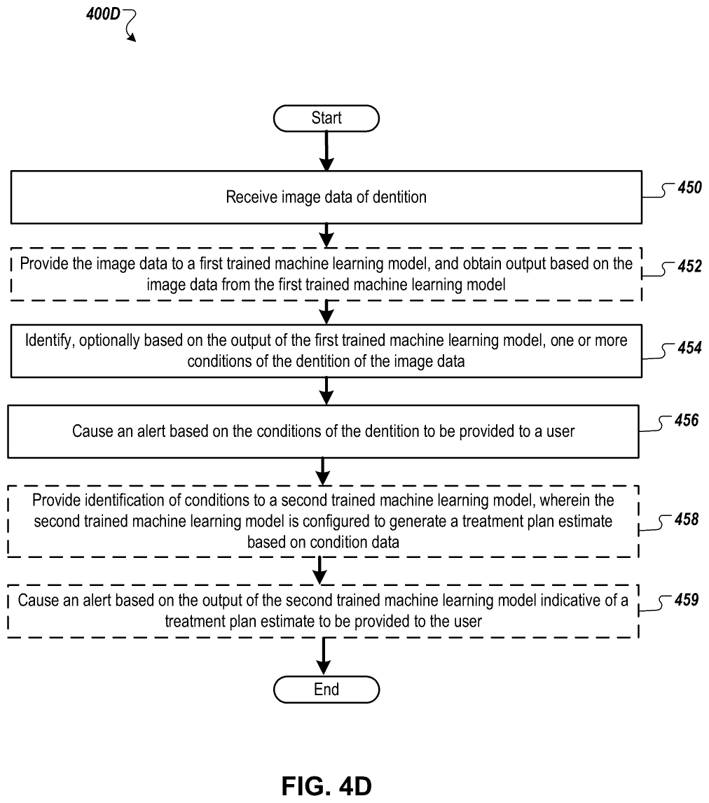

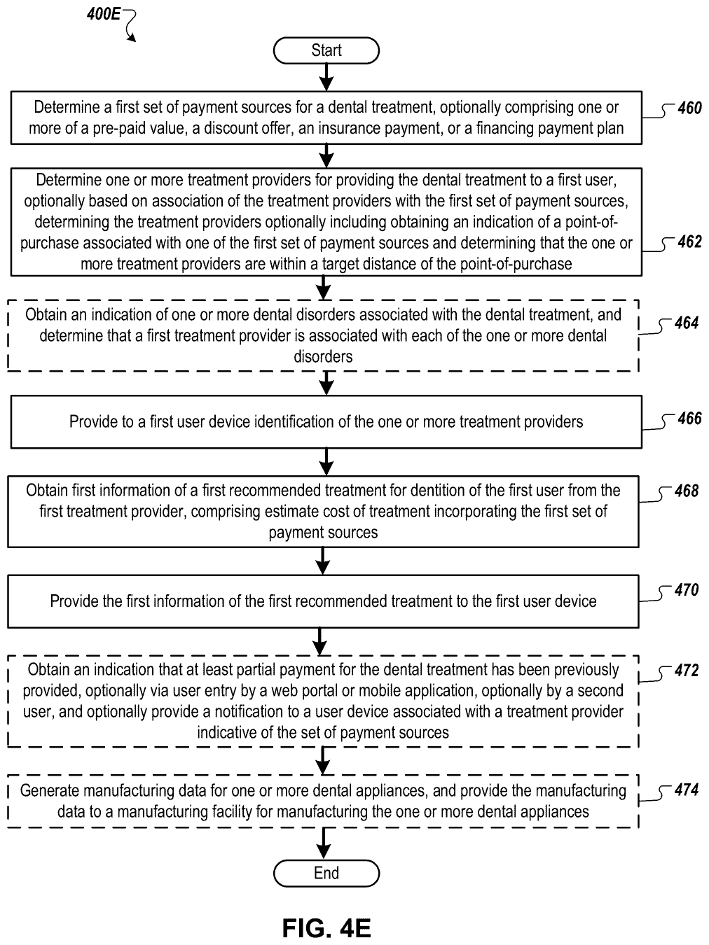

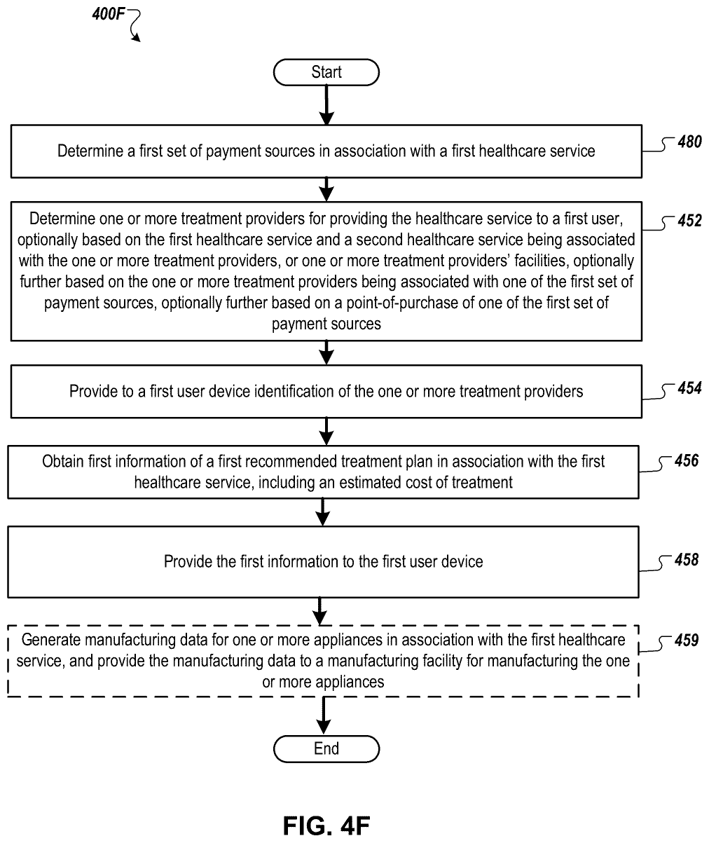

FIGS. 4A-F are flow diagrams of methods 400A-F associated with generating and providing treatment estimates, according to certain embodiments. Methods 400A-F may be performed by processing logic that may include hardware (e.g., circuitry, dedicated logic, programmable logic, microcode, processing device, etc.), software (such as instructions run on a processing device, a general purpose computer system, or a dedicated machine), firmware, microcode, or a combination thereof. In some embodiments, methods 400A-F may be performed, in part, by treatment communication system 102 of FIG. 1. Methods 400A-F may be performed by patient device 110A, or practitioner device 110B, in some embodiments. In some embodiments, a non-transitory machine-readable storage medium stores instructions that when executed by a processing device (e.g., of treatment communication system 102, of server machine 106, of patient device 110A or practitioner device 110B, etc.) cause the processing device to perform one or more of methods 400A-F.

For simplicity of explanation, methods 400A-F are depicted and described as a series of operations. However, operations in accordance with this disclosure can occur in various orders and/or concurrently and with other operations not presented and described herein. Furthermore, not all illustrated operations may be performed to implement methods 400A-F in accordance with the disclosed subject matter. In addition, those skilled in the art will understand and appreciate that methods 400A-F could alternatively be represented as a series of interrelated states via a state diagram or events.

FIG. 4A is a flow diagram of a method 400A for providing an estimate for treatment to a user, according to some embodiments. At block 402, processing logic determines a plurality of treatment providers for providing dental treatment to a user. The determination may be made by a server device, e.g., a backend device that manages data communicated with one or more client devices. The determination may be made responsive to receiving a request from a user device, a patient device, or the like. The request may be or include a request for one or more treatment providers. The plurality of treatment providers may be determined based on a location associated with the first user device (e.g., a location of the device, a location selected by a user via the first user device, or the like), a treatment history of the providers of the plurality of providers, a user rating of the providers, a type of disorder to be treated, information about the user (e.g., patient age), images provided by the user, a combination thereof, or the like.

At block 404, processing logic provides to a first user device identification of the plurality of treatment providers. The first user device may provide a notification to the user associated with receipt of the identification of the plurality of treatment providers. The plurality of treatment providers may be providers that are associated with a disorder of the user. The first user device may provide identification of the plurality of treatment providers for user review. The first user device may provide identification of the plurality of treatment providers for user selection. The first user device may receive selection from a user of one or more treatment providers that the user is interested in.

At block 405, processing logic receives, from the first user device, image data of dentition of the user. In some embodiments, the image data includes one or more two-dimensional (2D) color images. In some embodiments, the image data includes intraoral scans generated by an intraoral scanner and/or three-dimensional (3D) models of the user's upper and/or lower dental arches generated from intraoral scans. The image data may include one or more images of a dental arch of a user, which may or may not have been taken while the patient was wearing a cheek retractor. The image data may include indications of one or more disorders (e.g., or a first disorder). The first disorder may be a disorder associated with prosthodontic treatment, orthodontic treatment, or the like. The first disorder may be associated with one or more treatment providers of the plurality of treatment providers.

In some embodiments, processing logic processes the image data using one or more trained machine learning models that output dental condition estimations, which may include indications of one or more dental conditions and/or severity levels of one or more dental conditions.

At block 406, processing logic receives, from the first user device, a first user selection of a first treatment provider of the plurality of treatment providers. The first disorder (e.g., included in the image data of dentition) may be associated with the first treatment provider. For example, the first treatment provider may be qualified, trained, or the like to treat the first disorder, the first treatment provider may have a target amount of experience in treating the first disorder, or the like.

At block 407, processing logic further receives a second user selection of a second treatment provider of the plurality of treatment providers. Selection of the second treatment provider, identity of the second treatment provider, and the like may share one or more features with the first provider. In some embodiments, a user may experience multiple disorders, e.g., which may be treated by different treatment providers. In some embodiments, a second plurality of treatment providers, including a third treatment provider, may be provided to the first user device. In some embodiments, identification of a third treatment provider associated with a second disorder of the user may be provided to the first user device. In some embodiments, the images of dentition may further include indications of the second disorder. In some embodiments, the processing logic may further receive selection of the third treatment provider, in association with the second disorder.

At block 408, processing logic provides the image data and/or dental condition estimations to a second device association with the first treatment provider and a third device associated with the second treatment provider. The second device may perform functions of practitioner device 110B of FIG. 1. The treatment provider devices may present the image data (e.g., disorder data) and/or dental condition estimations to practitioners to enable the practitioners to generate treatment estimates. The treatment provider devices may further provide analysis (e.g., via one or more machine learning models) to assist the practitioners in generating treatment estimates. The treatment provider devices may provide disorder data to one or more models that predict case complexity, predict treatment steps, or the like. Alternatively, or additionally, analysis of the image data may be performed by a server computing device, and a result of the analysis may be presented to one or more of the practitioners. For example, one or more server computing devices may include one or more trained models that are trained to receive images of patient smiles (e.g., showing teeth), and to determine one or more disorders of the teeth (e.g., such as tooth crowding, malocclusions such as level 1, level 2 or level 3 malocclusions, overbite, underbite, cross bite, overjet, caries, tooth wear, gingival recession, gingival inflammation, etc.). In some embodiments, one or more of the analyses described in U.S. Publication No. 2022/0202295, published Jun. 30, 2022, and entitled Dental Diagnostics Hub, which is incorporated by reference herein, are performed to identify dental issues and/or optional treatments for those dental issues.

At block 410, processing logic obtains first information of a first recommended treatment for the dentition from the second device of the first treatment provider. Processing logic further obtains second information of a second recommended treatment for the dentition from the third device of the second treatment provider. The information of the recommended treatments may include estimated cost of treatment, estimated duration of treatment, estimate treatment steps (e.g., treatment tools, treatment techniques, number of stages of treatment, etc.), estimated treatment plans, or the like. The recommended treatments may include prosthodontic treatments, orthodontic treatments, and/or the like.

At block 412, processing logic provides the first information of the first recommended treatment and the second information of the second recommended treatment to the first user device. Providing the information of the recommended treatments may be performed responsive to receiving the information from a treatment provider's device. Processing logic may further receive, from the first user device, user selection of one of the recommended treatments based on the estimates. In some embodiments, processing logic may cause a device to alert a user or treatment provider to schedule a treatment appointment. In some embodiments, processing logic may cause a device to provide a UI element for scheduling treatment. In some embodiments, one or more messages may be received by processing logic and/or provided by the processing logic. For example, processing logic may receive a message from the first user device intended for receipt by the first treatment provider, and processing logic may provide the message to the second device associated with the first treatment provider.

FIG. 4B is a flow diagram of a method 400B for providing disorder data and receiving treatment estimates based on the disorder data, according to some embodiments. At block 420, processing logic obtains identification of a plurality of treatment providers. Obtaining identification may be performed by a user device, such as patient device 110A of FIG. 1. Obtaining identification may be responsive to obtaining a user request for identification of one or more treatment providers. Obtaining identification may be performed responsive to providing a request for identification of one or more treatment providers to a processing device, such as a server associated with a communication application.

At block 422, processing logic obtains user input indicative of a first disorder, first selection of a first treatment provider of the plurality of treatment providers, and second selection of a second treatment provider of the plurality of treatment providers. The user input indicative of the first disorder may include one or more images of a region, body part, or the like experiencing the disorder. The user input indicative of the first disorder (e.g., disorder data) may include images of dentition. The disorder data may include other forms of dentition data, such as x-ray data, computed tomography data, three-dimensional scan data, etc. In some embodiments, processing logic may further provide a prompt to the user to provide disorder data. The prompt may include providing data from an outside source (e.g., providing previously generated data). The prompt may include instructing the user to generate disorder data (e.g., generate one or more images). The prompt may include instructions. Processing logic may further provide one or more disorder images to one or more image assessment models, as described in connection with FIG. 3C. Output from the image assessment models may be obtained. Output from the image assessment models may include indications of one or more deficiencies of the disorder data, complexity of a disorder case, estimated treatment steps, or the like. In some embodiments, processing logic may provide an alert to a user based on image deficiencies, e.g., may prompt a user to adjust or replace one or more images.

Obtaining selection of a first and/or second treatment provider may be facilitated by further actions of the processing device. For example, a list, map, set, or other appearance of the plurality of treatment providers may be provided for a user to review. In some embodiments, the user may select one or more filters for the plurality of treatment providers, such as excluding treatment providers that do not satisfy one or more threshold conditions (e.g., threshold experience level, threshold maximum distance from the user, or the like). In some embodiments, the user may select a rearrangement of the treatment providers, e.g., users may be arranged by experience level, patient satisfaction level, distance from a target location, or another metric.

At block 424, processing logic provides the user input to a server device. The user input may further be provided to a practitioner device, e.g., associated with the first treatment provider.

At block 426, processing logic receives, from the server device, first information of a first recommended treatment in association with the first disorder and the first treatment provider, and second information of a second recommended treatment in association with the first disorder and the second treatment provider. The first and second information may include an estimated treatment cost. The first and second information may include an estimated treatment duration. The first and second information may include an estimated treatment plan. The first and second information may include estimated treatment techniques, appliances, tools, or the like.

At block 428, processing logic obtains a third selection of the first recommended treatment. The third selection may be provided by a user via a GUI. The third selection may indicate that the user intends to pursue the first recommended treatment. At block 429, processing logic provides an indication of the first recommended treatment based on the third selection of the first recommended treatment to the server device.

In some embodiments, responsive to the third selection, the processing logic may provide an appointment scheduling prompt to a user. The appointment scheduling prompt may include one or more elements for accepting an appointment proposed by the first treatment provider. The appointment scheduling prompt may include one or more elements for proposing an appointment to the first treatment provider. In some embodiments, an alert indicative of the appointment scheduling (e.g., the prompt, a successfully scheduled appointment, the appointment, or the like) may be provided to the user.

In some embodiments, processing logic may further obtain identification of a third treatment provider and a fourth treatment provider. The third and fourth treatment provider may be associated with a second disorder (e.g., a second disorder of the user). The identification of the third and fourth treatment providers may be provided to a user device responsive to receiving a request from the user device for identification of practitioners associated with the second disorder (e.g., practitioners who treat or have treated the second disorder). In some embodiments, user input indicative of the second disorder (e.g., disorder data, dentition images) may be provided, e.g., to devices associated with the third and fourth treatment providers. In some embodiments, third and fourth recommended treatments may be provided in association with the third and fourth treatment providers.

FIG. 4C is a flow diagram of a method 400C for providing recommended treatment information to a user, according to some embodiments. At block 430, processing logic obtains, from a first user device, a first user request for identification of a first treatment provider. Operations of method 400C may be performed by a server device, e.g., a device executing backend operations of a treatment estimate system, treatment communication system, or the like. At block 432, processing logic determines a treatment provider responsive to receiving the request. Determining the treatment provider may be based on a location associated with the first user device. Determining the treatment provider may be based on a treatment history of the treatment provider. Determining the treatment provider may be based on further information received from the first user device, such as information about a user, information regarding a disorder of the user, or the like. Determining the treatment provider may be based on user ratings of the treatment provider.

At block 434, processing logic provides identification of the treatment provider to the first user device. At block 436, processing logic receives, from the first user device, first user data. The first user data includes data indicative of dentition of the user (e.g., disorder data, disorder image data, dentition image data, etc.). The first user data includes a second user request for a first treatment plan associated with the dentition (e.g., the disorder) and the treatment provider. The request may be for the treatment provider to generate an estimate of a treatment based on the dentition data.

At block 438, processing logic provider the first user data to a second device (e.g., a practitioner device) associated with the treatment provider. At block 440, processing logic obtains, from the second device, information of a first recommended treatment in association with the first user data and the treatment provider. The information of the first recommended treatment may include an estimated cost of treatment. The information of the first recommended treatment may include an estimated duration of treatment. The information of the first recommended treatment may include one or more predicted aspects of a treatment plan. At block 442, processing logic provides to the first user device the information of the first recommended treatment. In some embodiments, a user may reject a recommended treatment, reject an estimate, or the like. Processing logic may receive a user rejection of the first recommended treatment. The user rejection may be provided to the second device associated with the treatment provider. The treatment provider via the second device may provide an updated estimate. The updated estimate (second recommended treatment) may be provided to the user device.

FIG. 4D is a flow diagram of a method 400D for generating treatment

estimates, according to some embodiments. In some embodiments, generating a treatment estimate may be performed by a treatment provider. In some embodiments, operations of generating a treatment estimate (e.g., to assist a treatment provider in providing an estimate) may be performed by a processing device, such as practitioner device 110B of FIG. 1. In some embodiments, some operations of generating a treatment estimate may be performed by one or more trained machine learning models, e.g., as described in connection with FIG. 3C. In some embodiments, operations of generating a treatment estimate may include or correspond to operations of providing virtual care to a patient, such as described in U.S. Publication No. 2022/0023003, published Jan. 27, 2022, which is incorporated by reference herein in its entirety.

At block 450, processing logic (e.g., of a practitioner device) receives image data of dentition. The image data may include dentition disorders. The image data may be provided from a user device, patient device, or the like. The image data may be received by multiple treatment providers, e.g., to enable a user to select between multiple treatment providers, multiple estimates, or the like. The image data may include 2D images (e.g., 2D color images), intraoral scans, 3D models, and so on.

At block 452, processing logic optionally provides image data to a first trained machine learning model. The first trained machine learning model may be configured to generate estimates of dentition conditions based on input images. In some embodiments, the first trained machine learning model may be configured to generate estimates of case complexity based on images of dentition, which may further be used in generating a treatment estimate (e.g., treatment cost estimate). The first trained machine learning model may be configured to generate predicted classifications of conditions of the dentition based on the image data, e.g., malocclusion, misalignment, or the like. In some embodiments, some case assessment operations may be performed by a server device, a user or patient device, or the like. Processing logic may optionally further obtain output from the first trained machine learning model based on the image data.

At block 454, processing logic identifies one or more conditions of the dentition of the image data. Processing logic may identify the one or more conditions based on output of the first trained machine learning model (e.g., output of the trained machine learning model ma include condition identification).

At block 456, processing logic causes an alert (e.g., a notification) based on the conditions of the dentition to be provided to a user (e.g., a treatment provider). The treatment provider may then utilize the conditions in generating one or more estimates of recommended treatment related to the dentition.

At block 458, processing logic optionally provides identification of conditions to a second trained machine learning model. The second trained machine learning model may be configured to generate one or more treatment plan estimates (e.g., estimated cost, duration, steps, techniques, or the like) based on the condition data. In some embodiments, image data may be provided to a model, and the model may generate a complexity score, condition identification, and/or predicted treatment estimates.

At block 459, processing logic optionally causes an alert based on the output of the second trained machine learning model indicative of a treatment plan estimate to be provided to the user (e.g., practitioner).

In some embodiments, processing logic performs one or more operations as described in U.S. Patent Publication No. 2022/0023003, published Jan. 27, 2023, which is incorporated by reference herein, for dental assessment, treatment tracking and/or reporting. Messages about such dental assessments, treatment tracking results and/or reporting may be sent between patient devices and practitioner devices using treatment communication system 102 of FIG. 1 in embodiments.

In some embodiments, a treatment planning data flow (e.g., of a communication application, executed by a web browser, or the like) may be responsible for generating a treatment plan estimate that includes a treatment outcome for a patient. The treatment plan may include and/or be based on one or more initial 2D and/or 3D images of a patient's dental arches. For example, the treatment planning application may receive 2D and/or 3D intraoral images (e.g., intraoral scans) of the patient's dental arches, and may stitch the images or scans together to create a virtual 3D model of the dental arches. Alternatively, the treatment planning application may receive a virtual 3D model of a patient's dental arches. In embodiments, the treatment planning application receives a patient record for a patient case, which may include, for example, intraoral scans, medical images, patient case details, 3D models of the patient's upper and/or lower dental arches, and so on.

The treatment planning method may then determine current positions and orientations of the patient's teeth from the virtual 3D model in a patient record and determine or receive target final positions and orientations for the patient's teeth represented as a treatment outcome. The treatment planning application may further determine one or more stages of treatment, and may determine target positions and orientations of the patient's teeth for each of the stages of treatment. The treatment planning application may additionally or alternatively determine one or more dental prosthetics to be used for a patient, such as a bridge, cap, crown, and so on. The treatment planning application may generate a case complexity metric based on the initial and/or final positions of various aspects of a patient's dentition. The complexity metric may be related to treatment practitioner choice, estimate of treatment cost, estimate of treatment duration, estimate of tools and/or techniques to be included in the treatment (e.g., mandibular advancement), or the like.

With respect to orthodontic treatment, the treatment planning application may generate a virtual 3D model showing the patient's dental arches at the end of treatment as well as one or more virtual 3D models showing the patient's dental arches at various intermediate stages of treatment. Alternatively, or additionally, the treatment planning application may generate one or more 3D images and/or 2D images showing the patient's dental arches at various stages of treatment. The 3D models for any of the steps of treatment may be manipulated using a medical computer aided drafting (CAD) application in embodiments.

By way of non-limiting example, a dental treatment outcome may be the result of a variety of dental procedures. Such dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions. The term prosthodontic procedure refers, inter alia, to any procedure involving the oral cavity and directed to the design, manufacture or installation of a dental prosthesis at a dental site within the oral cavity, or a real or virtual model thereof, or directed to the design and preparation of the dental site to receive such a prosthesis. A prosthesis may include any restoration such as implants, crowns, veneers, inlays, onlays, and bridges, for example, and any other artificial partial or complete denture. The term orthodontic procedure refers, inter alia, to any procedure involving the oral cavity and directed to the design, manufacture or installation of orthodontic elements at a dental site within the oral cavity, or a real or virtual model thereof, or directed to the design and preparation of the dental site to receive such orthodontic elements. These elements may be appliances including but not limited to brackets and wires, retainers, clear aligners, or functional appliances. Any of treatment outcomes or updates to treatment outcomes described herein may be based on these orthodontic and/or dental procedures. Examples of orthodontic treatments are treatments that reposition the teeth, treatments such as mandibular advancement that manipulate the lower jaw, treatments such as palatal expansion that widen the upper and/or lower palate, and so on. For example, an update to a treatment outcome may be generated by interaction with a user to perform one or more procedures to one or more portions of a patient's dental arch or mouth.

A treatment plan for producing a particular treatment outcome may be generated by first generating an intraoral scan of a patient's oral cavity. From the intraoral scan a virtual 3D model of the upper and/or lower dental arches of the patient may be generated. A dental practitioner may then determine a desired final position and orientation for the patient's teeth on the upper and lower dental arches, for the patient's bite, and so on. This information may be used by the treatment planning application to generate a virtual 3D model of the patient's upper and/or lower arches after orthodontic treatment. This data may be used to create an orthodontic treatment plan. The orthodontic treatment plan may include a sequence of orthodontic treatment stages. Each orthodontic treatment stage may adjust the patient's dentition by a prescribed amount, and may be associated with a 3D model of the patient's dental arch that shows the patient's dentition at that treatment stage.

In some embodiments, the treatment planning application may receive or generate one or more virtual 3D models, virtual 2D models, 3D images, 2D images, or other treatment outcome models and/or images, which may be based on intraoral images or scans. For example, an intraoral scan of the patient's oral cavity may have been performed to generate an initial virtual 3D model of the upper and/or lower dental arches of the patient. The treatment planning application may then determine a final treatment outcome based on the initial virtual 3D model, and then generate a new virtual 3D model representing the final treatment outcome. The treatment planning application may additionally determine various intermediate stages of orthodontic treatment, and generate virtual 3D models of the patient's dental arches for each such intermediate stage. Clinically important factors such as an amount of force to be applied to teeth, an amount of rotation to be achieved by teeth, an amount of movement of teeth, teeth interactions, and so on should be considered by the treatment planning application in generation of the intermediate treatment stages.

Once a treatment plan is finalized, the various 3D models of the patient's dental arches for each of the stages of treatment may be used to manufacture orthodontic aligners for each of the stages of treatment. The patient may then wear the orthodontic aligners in order for treatment. At the end of treatment, the patient should have corrected dentition.

FIG. 4E is a flow diagram of a method 400E for applying payment sources to a dental treatment, according to some embodiments. At block 460, process logic determines a first set of payment sources for a dental treatment. The first set of payment sources may include partial payment, partial pre-payment, or the like, e.g., in the form of a purchased gift card. The first set of payment sources may include one or more of a pre-paid value, a discount offer, an insurance payment, or a financing payment plan.

At block 462, process logic determines one or more treatment providers for providing the dental treatment to a first user. The first user may be a patient, potential patient, lead, or the like. The dental treatment may include orthodontic treatment, correction of malocclusion or misalignment, or the like. Determining the one or more treatment providers may be based on a location associated with the first user, e.g., a home address, a work address, a zipcode (e.g., a zipcode searched by the user), a point-of-sale of a gift card or other payment method, a point where a payment method was acquired (e.g., a promotional event), or the like. Determining the one or more treatment providers may be based on whether or not a treatment provider is associated with a payment source of the first set of payment sources (e.g., whether a practitioner has signed up for a program associated with a discount code, promotional event, or the like). Determining the one or more treatment providers may be based on a search or target location being within a threshold distance of a location of the treatment providers.

At block 464, process logic optionally obtains an indication of one or more dental disorders associated with the dental treatment, and determines that a first treatment provider is associated with each of the one or more dental disorders. The indication may include a submission describing the dental disorders by the first user. The indication may include one or more photos, videos, scans, x-rays, or other indications of dental properties of the first user. Process logic may further determine that a first treatment provider is qualified, experienced, or the like to provide dental treatment for a disorder of the first user. In some embodiments, the first user may exhibit more than one dental disorder, and a treatment provider may be recommended that is qualified to treat multiple of the dental disorders experienced by the first user, e.g., all the disorders of the first user.

At block 466, process logic provides to a first user device identification of the one or more treatment providers. The identification may include an indication of whether the one or more treatment providers accept the payment types of the first set of payment sources. The identification may include an indication of a treatment estimate (e.g., time of treatment, cost of treatment, etc.) by the one or more treatment providers.

At block 468, process logic obtains first information of a first recommended treatment for dentition of the first user from the treatment provider, comprising estimated cost of treatment incorporating the first set of payment sources.

At block 470, process logic provides the first information of the first recommended treatment to the first user device. For example, one or more details related to treatment of the dental disorder may be provided to the first user, enabling the first user to select a first treatment provider from the set of treatment providers. In some embodiments, operations related to blocks 466, 468, and 470 may result in a single alert being provided to a user device, e.g., an alert comprising a list of practitioners, their specialties, a list of treatment estimates, and a list of proposed treatment details.

At block 472, process logic optionally obtains an indication that at least partial payment for the dental treatment has been previously provided. For example, process logic may be provided an indication that a gift card has been purchased and/or registered with respect to a treatment of the first user. Optionally the indication may be provided to process logic by a user, e.g., the first user, via user entry of a web portal or mobile application. In some embodiments, a second user may have purchased and/or registered the payment source, e.g., as a gift to the first user. Process logic may further provide an alert to a user device associated with a treatment provider, e.g., a treatment provider selected by the first user. The alert may indicate a payment method, e.g., indicate that a portion of the cost of the treatment should be discounted, has already been paid, or the like.

At block 474, process logic optionally generates manufacturing data for one or more dental appliances, e.g., in connection with the first recommended treatment. Process logic may further provide the manufacturing data, e.g., to a manufacturing facility for manufacturing of the one or more dental appliances in connection with the first recommended treatment.

FIG. 4F is a flow diagram of a method 400F for applying payment sources to healthcare services, according to some embodiments. At block 480, process logic determines a first set of payment sources in association with a first healthcare service. The healthcare service may be or include one or more dental/orthodontic services. The first healthcare service may be a service unrelated to dental care, such as cosmetic surgery, botox, or other services that may benefit from operations of method 400F.

At block 452, process logic determines one or more treatment providers for providing the healthcare service to a first user. Determining the one or more healthcare treatment providers (e.g., service providers, practitioners, etc.) may be at least partially based on location of a healthcare service facility, location of a user, location of acquisition of one of the first set of payment sources, etc. In some embodiments (e.g., in the case of a user indicating multiple healthcare services), locations of multiple service providers may be considered. For example, a service provider that is proximate or shares a facility with another service provider that is also of relevance to the user seeking the first healthcare service may be identified. Determining the one or more treatment providers may further be based on the one or more treatment providers being associated with one or more of the set of payment sources (e.g., practitioners that have opted into a promotional program including a payment source, discount, or the like).

At block 454, process logic provides to a first user device (e.g., a patient device) identification of the one or more treatment providers. Operations of block 454 may share one or more features in common with operations of block 466 of FIG. 4E.

At block 456, process logic obtains first information of a first recommended treatment plan in association with the first healthcare service, including an estimated cost of treatment. At block 458, process logic provides the first information to the first user device. In some embodiments, operations of blocks 456 and 458 may be performed before operations of block 454, and the treatment estimates may be provided to the first user device along with the identification of the one or more treatment providers.

At block 459, process logic optionally generates manufacturing data for one or more appliances in association with the first healthcare service (e.g., three-dimensional models of appliances for treating one or more disorders). Optionally, process logic further provides the manufacturing data to a manufacturing facility for manufacturing the one or more appliances.

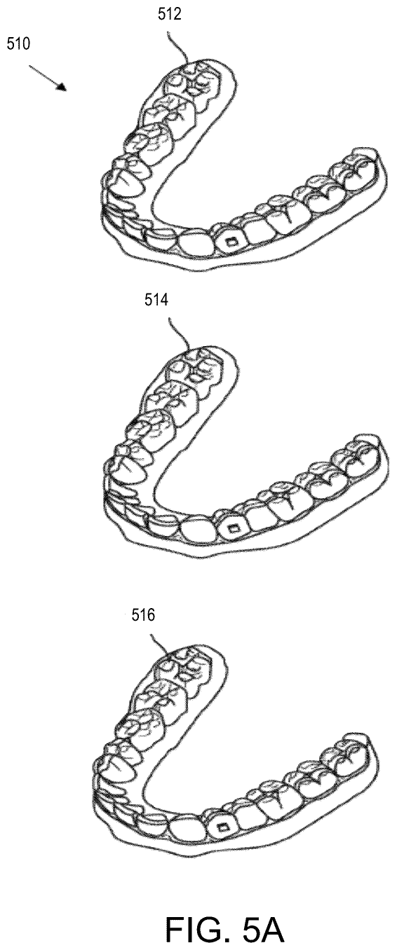

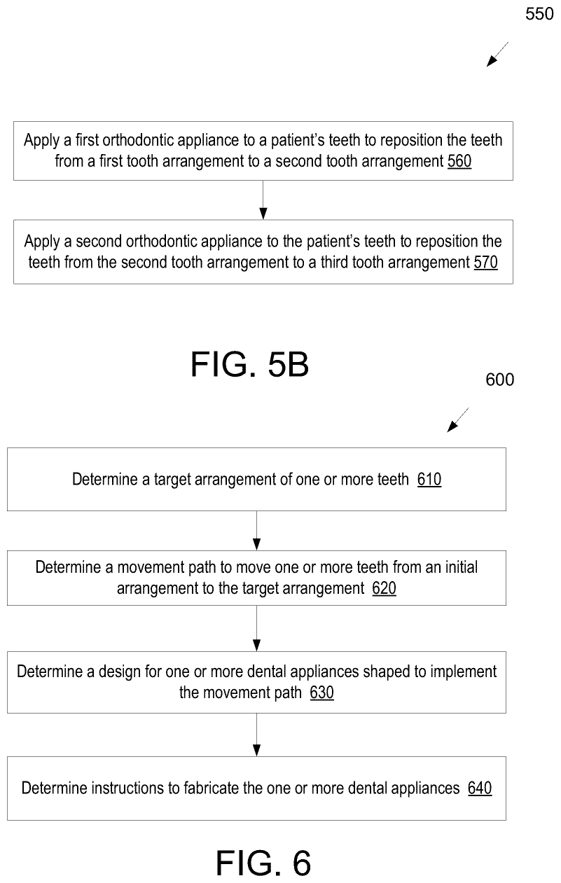

FIG. 5A illustrates a tooth repositioning system 510 including a plurality of appliances 512, 514, 516, which may be used to implement an orthodontic treatment plan recommended by a dental practitioner in embodiments. The appliances 512, 514, 516 can be designed based on generation of a sequence of 3D models of dental arches, which may be generated according to the techniques discussed herein above. Any of the appliances described herein can be designed and/or provided as part of a set of a plurality of appliances used in a tooth repositioning system, and may be designed in accordance with an orthodontic treatment plan generated in accordance with embodiments of the present disclosure. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 510 can include a first appliance 512 corresponding to an initial tooth arrangement, one or more intermediate appliances 514 corresponding to one or more intermediate arrangements, and a final appliance 516 corresponding to a target arrangement. A target tooth arrangement can be a planned final tooth arrangement selected for the patient's teeth at the end of all planned orthodontic treatment, as optionally output using a trained machine learning model. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

In some embodiments, the appliances 512, 514, 516 (or portions thereof) can be produced using indirect fabrication techniques, such as by thermoforming over a positive or negative mold. Indirect fabrication of an orthodontic appliance can involve producing a positive or negative mold of the patient's dentition in a target arrangement (e.g., by rapid prototyping, milling, etc.) and thermoforming one or more sheets of material over the mold in order to generate an appliance shell.

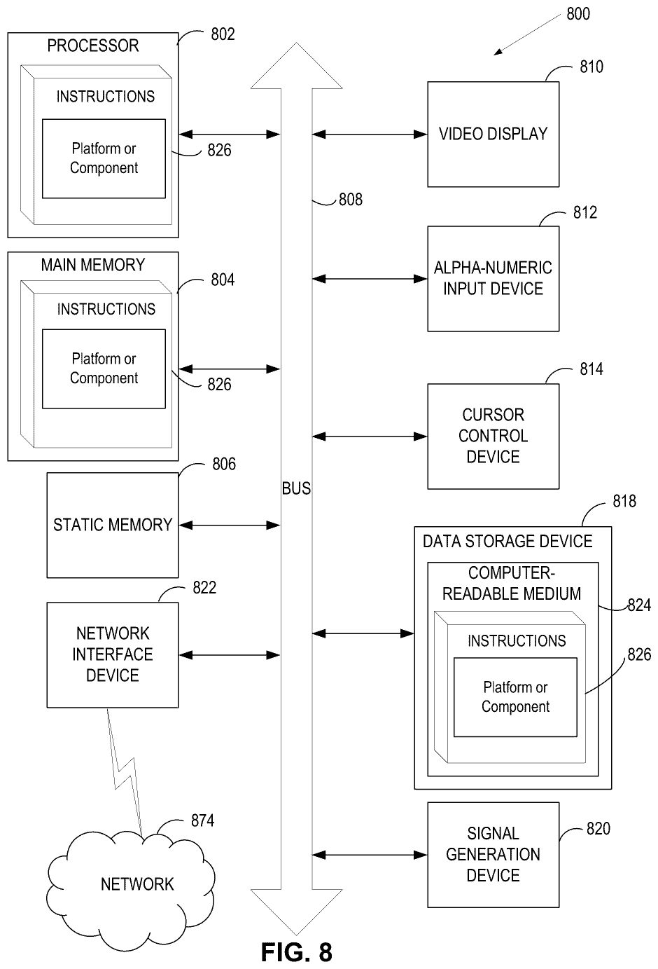

In an example of indirect fabrication, a mold of a patient's dental arch may be fabricated from a digital model of the dental arch generated by a trained machine learning model as described above, and a shell may be formed over the mold (e.g., by thermoforming a polymeric sheet over the mold of the dental arch and then trimming the thermoformed polymeric sheet). The fabrication of the mold may be performed by a rapid prototyping machine (e.g., a stereolithography (SLA) 3D printer). The rapid prototyping machine may receive digital models of molds of dental arches and/or digital models of the appliances 512, 514, 516 after the digital models of the appliances 512, 514, 516 have been processed by processing logic of a computing device, such as the computing device in FIG. 8. The processing logic may include hardware (e.g., circuitry, dedicated logic, programmable logic, microcode, etc.), software (e.g., instructions executed by a processing device), firmware, or a combination thereof. For example, one or more operations may be performed by a processing device executing dentition image assessment 368 of FIG. 3C.

To manufacture the molds, a shape of a dental arch for a patient at a treatment stage is determined based on a treatment plan. In the example of orthodontics, the treatment plan may be generated based on an intraoral scan of a dental arch to be modeled. The intraoral scan of the patient's dental arch may be performed to generate a three dimensional (3D) virtual model of the patient's dental arch (mold). For example, a full scan of the mandibular and/or maxillary arches of a patient may be performed to generate 3D virtual models thereof. The intraoral scan may be performed by creating multiple overlapping intraoral images from different scanning stations and then stitching together the intraoral images or scans to provide a composite 3D virtual model. In other applications, virtual 3D models may also be generated based on scans of an object to be modeled or based on use of computer aided drafting techniques (e.g., to design the virtual 3D mold). Alternatively, an initial negative mold may be generated from an actual object to be modeled (e.g., a dental impression or the like). The negative mold may then be scanned to determine a shape of a positive mold that will be produced.

Once the virtual 3D model of the patient's dental arch is generated, a dental practitioner may determine a desired treatment outcome, which includes final positions and orientations for the patient's teeth. In one embodiment, dental arch data generator 268 outputs a desired treatment outcome based on processing the virtual 3D model of the patient's dental arch (or other dental arch data associated with the virtual 3D model). Processing logic may then determine a number of treatment stages to cause the teeth to progress from starting positions and orientations to the target final positions and orientations. The shape of the final virtual 3D model and each intermediate virtual 3D model may be determined by computing the progression of tooth movement throughout orthodontic treatment from initial tooth placement and orientation to final corrected tooth placement and orientation. For each treatment stage, a separate virtual 3D model of the patient's dental arch at that treatment stage may be generated. In one embodiment, for each treatment stage dental arch data generator 268 outputs a different 3D model of the dental arch. The shape of each virtual 3D model will be different. The original virtual 3D model, the final virtual 3D model and each intermediate virtual 3D model is unique and customized to the patient.

Accordingly, multiple different virtual 3D models (digital designs) of a dental arch may be generated for a single patient. A first virtual 3D model may be a unique model of a patient's dental arch and/or teeth as they presently exist, and a final virtual 3D model may be a model of the patient's dental arch and/or teeth after correction of one or more teeth and/or a jaw. Multiple intermediate virtual 3D models may be modeled, each of which may be incrementally different from previous virtual 3D models.

Each virtual 3D model of a patient's dental arch may be used to generate a unique customized physical mold of the dental arch at a particular stage of treatment. The shape of the mold may be at least in part based on the shape of the virtual 3D model for that treatment stage. The virtual 3D model may be represented in a file such as a computer aided drafting (CAD) file or a 3D printable file such as a stereolithography (STL) file. The virtual 3D model for the mold may be sent to a third party (e.g., clinician office, laboratory, manufacturing facility or other entity). The virtual 3D model may include instructions that will control a fabrication system or device in order to produce the mold with specified geometries.

A clinician office, laboratory, manufacturing facility or other entity may receive the virtual 3D model of the mold, the digital model having been created as set forth above. The entity may input the digital model into a 3D printer. 3D printing includes any layer-based additive manufacturing processes. 3D printing may be achieved using an additive process, where successive layers of material are formed in proscribed shapes. 3D printing may be performed using extrusion deposition, granular materials binding, lamination, photopolymerization, continuous liquid interface production (CLIP), or other techniques. 3D printing may also be achieved using a subtractive process, such as milling.

In some instances, stereolithography (SLA), also known as optical fabrication solid imaging, is used to fabricate an SLA mold. In SLA, the mold is fabricated by successively printing thin layers of a photo-curable material (e.g., a polymeric resin) on top of one another. A platform rests in a bath of a liquid photopolymer or resin just below a surface of the bath. A light source (e.g., an ultraviolet laser) traces a pattern over the platform, curing the photopolymer where the light source is directed, to form a first layer of the mold. The platform is lowered incrementally, and the light source traces a new pattern over the platform to form another layer of the mold at each increment. This process repeats until the mold is completely fabricated. Once all of the layers of the mold are formed, the mold may be cleaned and cured.

Materials such as a polyester, a co-polyester, a polycarbonate, a polycarbonate, a thermopolymeric polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, a polytrimethylene terephthalate, a styrenic block copolymer (SBC), a silicone rubber, an elastomeric alloy, a thermopolymeric elastomer (TPE), a thermopolymeric vulcanizate (TPV) elastomer, a polyurethane elastomer, a block copolymer elastomer, a polyolefin blend elastomer, a thermopolymeric co-polyester elastomer, a thermopolymeric polyamide elastomer, or combinations thereof, may be used to directly form the mold. The materials used for fabrication of the mold can be provided in an uncured form (e.g., as a liquid, resin, powder, etc.) and can be cured (e.g., by photopolymerization, light curing, gas curing, laser curing, crosslinking, etc.). The properties of the material before curing may differ from the properties of the material after curing.

Appliances may be formed from each mold and when applied to the teeth of the patient, may provide forces to move the patient's teeth as dictated by the treatment plan. The shape of each appliance is unique and customized for a particular patient and a particular treatment stage. In an example, the appliances 512, 514, 516 can be pressure formed or thermoformed over the molds. Each mold may be used to fabricate an appliance that will apply forces to the patient's teeth at a particular stage of the orthodontic treatment. The appliances 512, 514, 516 each have teeth-receiving cavities that receive and resiliently reposition the teeth in accordance with a particular treatment stage.

In one embodiment, a sheet of material is pressure formed or thermoformed over the mold. The sheet may be, for example, a sheet of polymeric (e.g., an elastic thermopolymeric, a sheet of polymeric material, etc.). To thermoform the shell over the mold, the sheet of material may be heated to a temperature at which the sheet becomes pliable. Pressure may concurrently be applied to the sheet to form the now pliable sheet around the mold. Once the sheet cools, it will have a shape that conforms to the mold. In one embodiment, a release agent (e.g., a non-stick material) is applied to the mold before forming the shell. This may facilitate later removal of the mold from the shell. Forces may be applied to lift the appliance from the mold. In some instances, a breakage, warpage, or deformation may result from the removal forces. Accordingly, embodiments disclosed herein may determine where the probable point or points of damage may occur in a digital design of the appliance prior to manufacturing and may perform a corrective action.

Additional information may be added to the appliance. The additional information may be any information that pertains to the appliance. Examples of such additional information includes a part number identifier, patient name, a patient identifier, a case number, a sequence identifier (e.g., indicating which appliance a particular liner is in a treatment sequence), a date of manufacture, a clinician name, a logo and so forth. For example, after determining there is a probable point of damage in a digital design of an appliance, an indicator may be inserted into the digital design of the appliance. The indicator may represent a recommended place to begin removing the polymeric appliance to prevent the point of damage from manifesting during removal in some embodiments.

After an appliance is formed over a mold for a treatment stage, the appliance is removed from the mold (e.g., automated removal of the appliance from the mold), and the appliance is subsequently trimmed along a cutline (also referred to as a trim line). The processing logic may determine a cutline for the appliance. In one embodiment, a model (e.g., a trained machine learning model) outputs a cutline for an appliance associated with a 3D model. The determination of the cutline(s) may be made based on the virtual 3D model of the dental arch at a particular treatment stage, based on a virtual 3D model of the appliance to be formed over the dental arch, or a combination of a virtual 3D model of the dental arch and a virtual 3D model of the appliance. The location and shape of the cutline can be important to the functionality of the appliance (e.g., an ability of the appliance to apply desired forces to a patient's teeth) as well as the fit and comfort of the appliance. For shells such as orthodontic appliances, orthodontic retainers and orthodontic splints, the trimming of the shell may play a role in the efficacy of the shell for its intended purpose (e.g., aligning, retaining or positioning one or more teeth of a patient) as well as the fit of the shell on a patient's dental arch. For example, if too much of the shell is trimmed, then the shell may lose rigidity and an ability of the shell to exert force on a patient's teeth may be compromised. When too much of the shell is trimmed, the shell may become weaker at that location and may be a point of damage when a patient removes the shell from their teeth or when the shell is removed from the mold. In some embodiments, the cut line may be modified in the digital design of the appliance as one of the corrective actions taken when a probable point of damage is determined to exist in the digital design of the appliance.

On the other hand, if too little of the shell is trimmed, then portions of the shell may impinge on a patient's gums and cause discomfort, swelling, and/or other dental issues. Additionally, if too little of the shell is trimmed at a location, then the shell may be too rigid at that location. In some embodiments, the cutline may be a straight line across the appliance at the gingival line, below the gingival line, or above the gingival line. In some embodiments, the cutline may be a gingival cutline that represents an interface between an appliance and a patient's gingiva. In such embodiments, the cutline controls a distance between an edge of the appliance and a gum line or gingival surface of a patient.

Each patient has a unique dental arch with unique gingiva. Accordingly, the shape and position of the cutline may be unique and customized for each patient and for each stage of treatment. For instance, the cutline is customized to follow along the gum line (also referred to as the gingival line). In some embodiments, the cutline may be away from the gum line in some regions and on the gum line in other regions. For example, it may be desirable in some instances for the cutline to be away from the gum line (e.g., not touching the gum) where the shell will touch a tooth and on the gum line (e.g., touching the gum) in the interproximal regions between teeth. Accordingly, it is important that the shell be trimmed along a predetermined cutline.

FIG. 5B illustrates a method 550 of orthodontic treatment using a plurality of appliances, in accordance with embodiments. The method 550 can be practiced using any of the appliances or appliance sets described herein. Operations of method 550 may be estimated, planned, presented to a user, or the like, according to embodiments described herein, e.g., via a treatment communication system 102 of FIG. 1. In block 560, a first orthodontic appliance is applied to a patient's teeth in order to reposition the teeth from a first tooth arrangement to a second tooth arrangement. In block 570, a second orthodontic appliance is applied to the patient's teeth in order to reposition the teeth from the second tooth arrangement to a third tooth arrangement. The method 550 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

FIG. 6 illustrates a method 600 for designing an orthodontic appliance to be produced by direct or indirect fabrication, in accordance with embodiments. The method 600 can be applied to any embodiment of the orthodontic appliances described herein, and may be performed using one or more trained machine learning models in embodiments. Some or all of the blocks of the method 600 can be performed by any suitable data processing system or device, e.g., one or more processors configured with suitable instructions.

At block 610 a target arrangement of one or more teeth of a patient may be determined. The target arrangement of the teeth (e.g., a desired and intended end result of orthodontic treatment) can be received from a clinician in the form of a prescription, can be calculated from basic orthodontic principles, can be extrapolated computationally from a clinical prescription, and/or can be generated by a trained machine learning model. With a specification of the desired final positions of the teeth and a digital representation of the teeth themselves, the final position and surface geometry of each tooth can be specified to form a complete model of the tooth arrangement at the desired end of treatment.

In block 620, a movement path to move the one or more teeth from an initial arrangement to the target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. An initial arrangement may be estimated by projecting some measurement of the patient's teeth to a latent space, and obtaining from the latent space a representation of the initial arrangement. From the obtained data, a digital data set such as a 3D model of the patient's dental arch or arches can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, optionally including measured or extrapolated hidden surfaces and root structures, as well as surrounding bone and soft tissue.

Having both an initial position and a target position for each tooth, a movement path can be defined for the motion of each tooth. Determining the movement path for one or more teeth may include identifying a plurality of incremental arrangements of the one or more teeth to implement the movement path. In some embodiments, the movement path implements one or more force systems on the one or more teeth (e.g., as described below). In some embodiments, movement paths are determined by a trained machine learning model such as treatment plan generator 276. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In some embodiments, a force system to produce movement of the one or more teeth along the movement path is determined. In one embodiment, the force system is determined by a trained machine learning model. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully-formed suture. Thus, in juvenile patients and others without fully-closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be considered. In other embodiments, the treating professional can select an appropriate treatment based on physiological characteristics of the patient. For example, the properties of the palate may also be estimated based on factors such as the patient's age-for example, young juvenile patients will typically require lower forces to expand the suture than older patients, as the suture has not yet fully formed.

In block 630, a design for one or more dental appliances shaped to implement the movement path is determined. In one embodiment, the one or more dental appliances are shaped to move the one or more teeth toward corresponding incremental arrangements. In some embodiments, results of one or more stages of treatment may be predicted by a machine learning model, such as a treatment prediction model described in connection with FIG. 3C. Determination of the one or more dental or orthodontic appliances, appliance geometry, material composition, and/or properties can be performed using a treatment or force application simulation environment. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA (Abaqus) software products from Dassault Systèmes of Waltham, MA.

In block 640, instructions for fabrication of the one or more dental appliances are determined or identified. In some embodiments, the instructions identify one or more geometries of the one or more dental appliances. In some embodiments, the instructions identify slices to make layers of the one or more dental appliances with a 3D printer. In some embodiments, the instructions identify one or more geometries of molds usable to indirectly fabricate the one or more dental appliances (e.g., by thermoforming plastic sheets over the 3D printed molds). The dental appliances may include one or more of aligners (e.g., orthodontic aligners), retainers, incremental palatal expanders, attachment templates, and so on.

In one embodiment, instructions for fabrication of the one or more dental appliances are generated by a trained model. In some embodiments, predictions of treatment progression and/or treatment appliances may be performed and/or aided by one or more models (e.g., trained machine learning models). The instructions can be configured to control a fabrication system or device in order to produce the orthodontic appliance with the specified orthodontic appliance. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by 3D printing a mold and thermoforming a plastic sheet over the mold.

Method 600 may comprise additional blocks: 1) The upper arch and palate of the patient is scanned intraorally to generate three dimensional data of the palate and upper arch; 2) The three dimensional shape profile of the appliance is determined to provide a gap and teeth engagement structures as described herein.

Although the above blocks show a method 600 of designing an orthodontic appliance in accordance with some embodiments, a person of ordinary skill in the art will recognize some variations based on the teaching described herein. Some of the blocks may comprise sub-blocks. Some of the blocks may be repeated as often as desired. One or more blocks of the method 600 may be performed with any suitable fabrication system or device, such as the embodiments described herein. Some of the blocks may be optional, and the order of the blocks can be varied as desired.

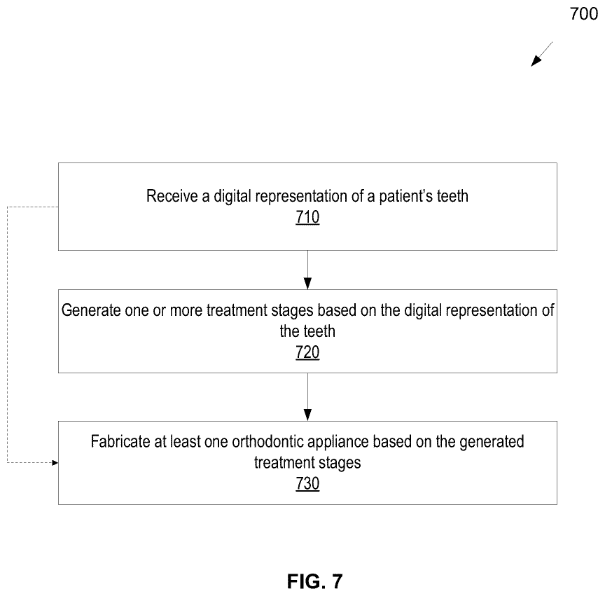

FIG. 7 illustrates a method 700 for digitally planning an orthodontic treatment and/or design or fabrication of an appliance, in accordance with embodiments. The method 700 can be applied to any of the treatment procedures described herein and can be performed by any suitable data processing system.

In block 710, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In block 720, one or more treatment stages are generated based on the digital representation of the teeth. In some embodiments, the one or more treatment stages are generated based on processing of input dental arch data by a trained machine learning model such as a treatment prediction model described in connection with FIG. 3C. Each treatment stage may include a generated 3D model of a dental arch at that treatment stage. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In block 730, at least one orthodontic appliance is fabricated based on the generated treatment stages. For example, a set of appliances can be fabricated, each shaped according to a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired. The fabrication of the appliance may include automated removal of the appliance from a mold (e.g., automated removal of an untrimmed shell from mold a using a shell removal device).

In some instances, staging of various arrangements or treatment stages may not be necessary for design and/or fabrication of an appliance. As illustrated by the dashed line in FIG. 7, design and/or fabrication of an orthodontic appliance, and perhaps a particular orthodontic treatment, may include use of a representation of the patient's teeth (e.g., receive a digital representation of the patient's teeth at block 710), followed by design and/or fabrication of an orthodontic appliance based on a representation of the patient's teeth in the arrangement represented by the received representation.

FIG. 8 is a block diagram illustrating a computer system 800, according to some embodiments. In some embodiments, computer system 800 may be connected (e.g., via a network, such as a Local Area Network (LAN), an intranet, an extranet, or the Internet) to other computer systems. Computer system 800 may operate in the capacity of a server or a client computer in a client-server environment, or as a peer computer in a peer-to-peer or distributed network environment. Computer system 800 may be provided by a personal computer (PC), a tablet PC, a Set-Top Box (STB), a Personal Digital Assistant (PDA), a cellular telephone, a web appliance, a server, a network router, switch or bridge, or any device capable of executing a set of instructions (sequential or otherwise) that specify actions to be taken by that device. Further, the term “computer” shall include any collection of computers that individually or jointly execute a set (or multiple sets) of instructions to perform any one or more of the methods described herein.

In a further aspect, the computer system 800 may include a processing device 802, a volatile memory 804 (e.g., Random Access Memory (RAM)), a non-volatile memory 806 (e.g., Read-Only Memory (ROM) or Electrically-Erasable Programmable ROM (EEPROM)), and a data storage device 818, which may communicate with each other via a bus 808.

Processing device 802 may be provided by one or more processors such as a general purpose processor (such as, for example, a Complex Instruction Set Computing (CISC) microprocessor, a Reduced Instruction Set Computing (RISC) microprocessor, a Very Long Instruction Word (VLIW) microprocessor, a microprocessor implementing other types of instruction sets, or a microprocessor implementing a combination of types of instruction sets) or a specialized processor (such as, for example, an Application Specific Integrated Circuit (ASIC), a Field Programmable Gate Array (FPGA), a Digital Signal Processor (DSP), or a network processor.

Computer system 800 may further include a network interface device 822 (e.g., coupled to network 874). Computer system 800 also may include a video display unit 810 (e.g., an LCD), an alphanumeric input device 812 (e.g., a keyboard), a cursor control device 814 (e.g., a mouse), and a signal generation device 820.

In some embodiments, data storage device 818 may include a non-transitory computer-readable storage medium 824 (e.g., non-transitory machine-readable medium) on which may store instructions 826 encoding any one or more of the methods or functions described herein, including instructions encoding components of FIG. 1 (e.g., various platforms of treatment communication system 102, communication applications 115, components of communication applications 115 such as messaging components 114, etc.) and for implementing methods described herein.

Instructions 826 may also reside, completely or partially, within volatile memory 804 and/or within processing device 802 during execution thereof by computer system 800, hence, volatile memory 804 and processing device 802 may also constitute machine-readable storage media.

While computer-readable storage medium 824 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein. The term “computer-readable storage medium” shall include, but not be limited to, solid-state memories, optical media, and magnetic media.

The methods, components, and features described herein may be implemented by discrete hardware components or may be integrated in the functionality of other hardware components such as ASICS, FPGAs, DSPs or similar devices. In addition, the methods, components, and features may be implemented by firmware modules or functional circuitry within hardware devices. Further, the methods, components, and features may be implemented in any combination of hardware devices and computer program components, or in computer programs.

Unless specifically stated otherwise, terms such as “receiving,” “performing,” “providing,” “obtaining,” “causing,” “accessing,” “determining,” “adding,” “using,” “training,” “reducing,” “generating,” “correcting,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices. Also, the terms “first,” “second,” “third,” “fourth,” etc. as used herein are meant as labels to distinguish among different elements and may not have an ordinal meaning according to their numerical designation.

Examples described herein also relate to an apparatus for performing the methods described herein. This apparatus may be specially constructed for performing the methods described herein, or it may include a general purpose computer system selectively programmed by a computer program stored in the computer system. Such a computer program may be stored in a computer-readable tangible storage medium.

The methods and illustrative examples described herein are not inherently related to any particular computer or other apparatus. Various general purpose systems may be used in accordance with the teachings described herein, or it may prove convenient to construct more specialized apparatus to perform methods described herein and/or each of their individual functions, routines, subroutines, or operations. Examples of the structure for a variety of these systems are set forth in the description above.

The above description is intended to be illustrative, and not restrictive. Although the present disclosure has been described with references to specific illustrative examples and embodiments, it will be recognized that the present disclosure is not limited to the examples and embodiments described. The scope of the disclosure should be determined with reference to the following claims, along with the full scope of equivalents to which the claims are entitled.

1. A method comprising:
determining, by a processing device, a plurality of treatment providers for providing a dental treatment to a user;
providing to a first user device identification of the plurality of treatment providers;
receiving, from the first user device:
image data of dentition of the user,
a first user selection of a first treatment provider of the plurality of treatment providers, and
a second user selection of a second treatment provider of the plurality of treatment providers;
providing the image data to a second device associated with the first treatment provider and a third device associated with the second treatment provider;
obtaining, by the processing device:
first information of a first recommended treatment for the dentition from the second device of the first treatment provider, and
second information of a second recommended treatment for the dentition from the third device of the second treatment provider; and
providing the first information of the first recommended treatment and the second information of the second recommended treatment to the first user device.

2. The method of claim 1, further comprising receiving, from the first user device, a request for identification of one or more treatment providers, wherein determining the plurality of treatment providers is performed responsive to the request.

3. The method of claim 2, wherein determining the plurality of treatment providers is based on one or more of:
a location associated with the first user device;
a treatment history of the first treatment provider; or
a user rating of the first treatment provider.

4. The method of claim 1, wherein the first information of the first recommended treatment comprises one or more of an estimated cost of treatment or estimated duration of treatment.

5. The method of claim 1, wherein the first recommended treatment comprises one or more of prosthodontic treatment or orthodontic treatment.

6. The method of claim 1, wherein the image data of dentition of the user comprises one or more images of a dental arch of a user comprising an indication of a first disorder associated with the first treatment provider and the second treatment provider.

7. The method of claim 6, further comprising:
determining a third treatment provider associated with a second disorder, wherein the image data of dentition of the user further comprises an indication of the second disorder; and
providing to the first user device identification of the third treatment provider.

8. The method of claim 1, further comprising obtaining, from the first user device, a third user selection of first recommended treatment.

9. The method of claim 8, further comprising prompting one or more of the user associated with the first user device, or the first treatment provider, to schedule treatment.

10. The method of claim 1, further comprising receiving, via the first user device, a message for the first treatment provider, and providing the message to the second device associated with the first treatment provider.

11. A method comprising:
obtaining, by a processing device, identification of a plurality of treatment providers;
obtaining user input indicative of:
a first disorder,
first selection of a first treatment provider of the plurality of treatment providers, and
second selection of a second treatment provider of the plurality of treatment providers;
providing the user input to a server device;
receiving, from the server device:
first information of a first recommended treatment in association with the first disorder and the first treatment provider, and
second information of a second recommended treatment in association with the first disorder and the second treatment provider;
obtaining a third selection of the first recommended treatment; and
providing an indication of the first recommended treatment based on the third selection of the first recommended treatment to the server device.

12. The method of claim 11, further comprising:
obtaining a user request for identification of one or more treatment providers; and
providing an indication of the user request to a server device, wherein the identification of the plurality of treatment providers is based on the user request.

13. The method of claim 11, wherein the first information of the first recommended treatment comprises one or more of an estimated cost of treatment or estimated duration of treatment.

14. The method of claim 11, further comprising prompting a user to generate one or more images of dentition of the user, wherein the user input indicative of the first disorder comprises the one or more images of the dentition.

15. The method of claim 14, further comprising:
obtaining a first image of dentition of the user;
providing the first image to an image assessment model as input;
obtaining from the image assessment model an indication of a deficiency of the first image of the dentition; and
providing an alert to the user based on the deficiency of the first image.

16. The method of claim 11, further comprising obtaining treatment scheduling data associated with the first recommended treatment, and providing an alert based on the treatment scheduling data to the user.

17. The method of claim 11, further comprising:
obtaining identification of a third treatment provider and a fourth treatment provider, wherein the third treatment provider and the fourth treatment provider are associated with a second disorder;
obtaining user input indicative of the second disorder;
providing to the server device the user input; and
obtaining third information of a third recommended treatment associated with the second disorder and the third treatment provider, and fourth information of a fourth recommended treatment in association with the second disorder and the fourth treatment provider.

18. A method, comprising:
obtaining, by a processing device, from a first user device, a first user request for identification of a first treatment provider;
determining, by the processing device, a treatment provider;
providing identification of the treatment provider to the first user device;
receiving, from the first user device, first user data comprising:
data indicative of dentition of the user, and
a second user request for a first treatment plan associated with the dentition and the treatment provider;
providing the first user data to a second device associated with the treatment provider;
obtaining, from the second device, information of a first recommended treatment in association with the first user data and the treatment provider; and
providing to the first user device the information of the first recommended treatment.

19. The method of claim 18, further comprising:
obtaining second user data comprising a user rejection of the first recommended treatment;
providing the user rejection to the second device;
obtaining a second recommended treatment from the second device; and
providing data indicative of the second recommended treatment to the first user device.

20. The method of claim 18, wherein determining the treatment provider is based on one or more of:
a location associated with the user device;
a treatment history of the treatment provider; or a user rating of the treatment provider.

21. The method of claim 18, wherein the information of the first recommended treatment comprises one or more of an estimated cost or estimated duration of treatment.

22.-38. (canceled)
